(19) United States
(12) Patent Application Publication
(10) Pub. No.: US 2020/0155625 A1
(43) Pub. Date: May 21, 2020

(54) ANTI-ANGIOGENIC ADENOVIRUS

(71) Applicant: EpicentRx, Inc., La Jolla, CA (US)

(72) Inventors: Christopher Larson, San Diego, CA (US); Tony R. Reid, San Diego, CA (US); Bryan T. Oronsky, Los Altos Hills, CA (US)

(21) Appl. No.: 16/616,146

(22) PCT Filed: May 24, 2018
(86) PCT No.: PCT/US2018/034487

§ 371 Date: Nov. 22, 2019

Related U.S. Application Data
(60) Provisional application No. 62/514,351, filed on Jun. 2, 2017.
(60) Provisional application No. 62/510,647, filed on May 24, 2017.

(51) Int. Cl. A61K 35/761; C12N 7/00; C12N 15/86; A61K 39/395; A61P 35/00

(52) U.S. Cl. CPC A61K 35/761; C12N 7/00; C12N 15/86; C12N 2710/00032; A61P 35/00; C12N 2710/10021; C12N 2710/00043; A61K 39/3955

(57) ABSTRACT

The invention relates to a recombinant adenovirus that expresses endostatin, angiostatin, or a combination of endostatin and angiostatin. The invention also relates to method of treating cancer in a subject in need thereof, the method comprising administering to the subject an effective amount of a combination of (i) a recombinant adenovirus and (ii) an anti-angiogenic agent to treat the cancer in the subject.

### CROSS-REFERENCE TO RELATED APPLICATIONS

This application claims the benefit of, and priority to, U.S. Provisional Patent Application Ser. No. 62/510,647, filed May 24, 2017 and U.S. Provisional Patent Application Ser. No. 62/514,351, filed Jun. 2, 2017.

### FIELD OF THE INVENTION

The field of the invention is molecular biology and virology, specifically recombinant adenoviruses and methods of treating subjects using recombinant adenoviruses.

### BACKGROUND

Despite extensive knowledge of the underlying molecular mechanisms that cause cancer, most advanced cancers remain incurable with current chemotherapy and radiation protocols. Oncolytic viruses have emerged as a platform technology that has the potential to significantly augment current standard treatment for a variety of malignancies (Kumar, S. et al. (2008) CURRENT OPINION IN MOLECULAR THERAPEUTICS 10(4):371-379; Kim, D. (2001) EXPERT OPINION ON BIOLOGICAL THERAPY 1(3):525-538; Kim D. (2000) ONCOGENE 19(56):6660-6669). These viruses have shown promise as oncolytic agents that not only directly destroy malignant cells via an infection-to-reproduction-to-lysis chain reaction but also indirectly induce anti-tumor immunity. These immune stimulatory properties have been augmented with the insertion of therapeutic transgenes that are copied and expressed each time the virus replicates.

Previously developed oncolytic viruses include the oncolytic serotype 5 adenovirus referred to as TAV-255 that is transcriptionally attenuated in normal cells but transcriptionally active in cancer cells (see, PCT Publication No. WO2010/101921). It is believed that the mechanism by which the TAV-255 vector achieves such tumor selectivity is through targeted deletion of three transcriptional factor (TF) binding sites for the transcription factors Pea3 and E2F, proteins that regulate adenovirus expression of E1a, the earliest gene to be transcribed after virus entry into the host cell, through binding to specific DNA sequences.

Despite the efforts to date, there is a need for improved oncolytic viruses for treating human subjects.

### SUMMARY OF THE INVENTION

The invention is based, in part, upon the discovery of recombinant adenoviruses that can efficiently express anti-angiogenic factors such as endostatin and/or angiostatin. Additionally, the invention is based, in part, upon the discovery that an anti-cancer treatment using an anti-VEGF antibody, e.g., bevacizumab, can be enhanced when the anti-VEGF antibody is administered in combination with a recombinant adenovirus, e.g., an endostatin and/or angiostatin expressing adenovirus described herein. Surprisingly, it has been discovered that for certain cancers, the recombinant adenoviruses described herein, administered alone or in combination with an anti-VEGF antibody, e.g., bevacizumab, do not merely slow or stop cancer growth but cause a cancer to go in to partial and/or complete remission.

Accordingly, in one aspect, the invention provides a recombinant adenovirus comprising a first nucleotide sequence encoding a first therapeutic transgene selected from endostatin and angiostatin inserted into an E1b-19K insertion site; wherein the E1b-19K insertion site is located between the start site of E1b-19K and the start site of E1b-55K.

In certain embodiments, the recombinant adenovirus is a type 5 adenovirus (Ad5).

In certain embodiments, the E1b-19K insertion site is located between the start site of E1b-19K and the stop site of E1b-19K. In certain embodiments, the E1b-19K insertion site comprises a deletion of from about 100 to about 305, about 100 to about 300, about 100 to about 250, about 100 to about 200, about 100 to about 150, about 150 to about 305, about 150 to about 300, about 150 to about 250, or about 150 to about 200 nucleotides adjacent the start site of E1b-19K. In certain embodiments, the E1b-19K insertion site comprises a deletion of about 200 nucleotides, e.g., 202 or 203 nucleotides adjacent the start site of E1b-19K. In certain embodiments, the E1b-19K insertion site comprises a deletion corresponding to nucleotides 1714-1916 of the Ad5 genome (SEQ ID NO: 1), or the first therapeutic transgene is inserted between nucleotides corresponding to 1713 and 1917 of the Ad5 genome (SEQ ID NO: 1). In certain embodiments, the first therapeutic transgene is inserted between CTGACCTC (SEQ ID NO: 2) and TCACCAGG (SEQ ID NO: 3), e.g., the recombinant adenovirus comprises, in a 5′ to 3′ orientation, CTGACCTC (SEQ ID NO: 2), the first therapeutic transgene, and TCACCAGG (SEQ ID NO: 3).

In certain embodiments, the recombinant adenovirus comprises a second nucleotide sequence encoding a second therapeutic transgene selected from endostatin and angiostatin. In certain embodiments, the second therapeutic transgene is inserted into the E1b-19k insertion site, and the first nucleotide sequence and the second nucleotide sequence are separated by an internal ribosome entry site (IRES). The IRES may, e.g., be selected from an encephalomyocarditis virus (EMCV) IRES, a foot-and-mouth disease virus (FMDV) IRES, and a poliovirus IRES. The IRES may, e.g., be an encephalomyocarditis virus (EMCV) IRES, e.g., the IRES may comprise SEQ ID NO: 20. In certain embodiments, the first and second therapeutic transgenes are inserted between nucleotides corresponding to 1713 and 1917 of the Ad5 genome (SEQ ID NO: 1), e.g., the first and second therapeutic transgenes are inserted between CTGACCTC (SEQ ID NO: 2) and TCACCAGG (SEQ ID NO: 3), e.g., the recombinant adenovirus comprises, in a 5′ to 3′ orientation, CTGACCTC (SEQ ID NO: 2), the first therapeutic transgene, the IRES, the second therapeutic transgene, and TCACCAGG (SEQ ID NO: 3).

In certain embodiments, the recombinant adenovirus comprises an E3 deletion. In certain embodiments, the E3 deletion comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or from about 3000 to about 3185 nucleotides. In certain embodiments, the E3 deletion site is located between the stop site of pVIII and the start site of Fiber. In certain embodiments, the E3 deletion site is located between the stop site of E3-10.5K and the stop site of E3-14.7K. In certain embodiments, the E3 deletion comprises a deletion of from about 500 to about 1551, from about 500 to about 1500, from about 500 to about 1000, from about 1000 to about 1551, from about 1000 to about 1500, or from about 1500 to about 1551 nucleotides adjacent the stop site of E3-10.5K. In certain embodiments, the E3 deletion comprises a deletion of about 1050 nucleotides adjacent the stop site of E3-10.5K, e.g., the E3 deletion comprises a deletion of 1063 or 1064 nucleotides adjacent the stop site of E3-10.5K. In certain embodiments, the E3 deletion comprises a deletion corresponding to the Ad5 dl309 E3 deletion. In certain embodiments, the E3 deletion comprises a deletion corresponding to nucleotides 29773-30836 of the Ad5 genome (SEQ ID NO: 1).

In certain embodiments, the second therapeutic transgene is inserted into an E3 insertion site, wherein the E3 insertion site is located between the stop site of pVIII and the start site of Fiber. In certain embodiments, the E3 insertion site comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or from about 3000 to about 3185 nucleotides. In certain embodiments, the E3 insertion site is located between the stop site of E3-10.5K and the stop site of E3-14.7K. In certain embodiments, the E3 insertion site comprises a deletion of from about 500 to about 1551, from about 500 to about 1500, from about 500 to about 1000, from about 1000 to about 1551, from about 1000 to about 1500, or from about 1500 to about 1551 nucleotides adjacent the stop site of E3-10.5K. In certain embodiments, the E3 insertion site comprises a deletion of about 1050 nucleotides adjacent the stop site of E3-10.5K, e.g., the E3 insertion site comprises a deletion of 1063 or 1064 nucleotides adjacent the stop site of E3-10.5K. In certain embodiments, the E3 insertion site comprises a deletion corresponding to the Ad5 dl309 E3 deletion. In certain embodiments, the E3 insertion site comprises a deletion corresponding to nucleotides 29773-30836 of the Ad5 genome (SEQ ID NO: 1), or the second therapeutic transgene is inserted between nucleotides corresponding to 29773 and 30836 of the Ad5 genome (SEQ ID NO: 1). In certain embodiments, the second therapeutic transgene is inserted between CAGTATGA (SEQ ID NO: 4) and TAATAAAAAA (SEQ ID NO: 5), e.g., the recombinant adenovirus comprises, in a 5′ to 3′ orientation, CAGTATGA (SEQ ID NO: 4), the second therapeutic transgene, and TAATAAAAAA (SEQ ID NO: 5).

In certain embodiments, in any of the foregoing adenoviruses, the recombinant adenovirus comprises a nucleotide sequence encoding the amino acid sequence of SEQ ID NO: 7 or SEQ ID NO: 8, or a sequence having 80%, 85%, 86%, 87%, 88%, 89%, 90%, 91%, 92%, 93%, 94%, 95%, 96%, 97%, 98%, or 99% sequence identity to SEQ ID NO: 7 or SEQ ID NO: 8. In certain embodiments, in any of the foregoing adenoviruses, the recombinant adenovirus comprises the nucleotide sequence of SEQ ID NO: 9 or SEQ ID NO: 10, or a sequence having 80%, 85%, 86%, 87%, 88%, 89%, 90%, 91%, 92%, 93%, 94%, 95%, 96%, 97%, 98%, or 99% sequence identity to SEQ ID NO: 9 or SEQ ID NO: 10.

In certain embodiments, in any of the foregoing adenoviruses, the recombinant adenovirus comprises a nucleotide sequence encoding the amino acid sequence of SEQ ID NO: 12, SEQ ID NO: 13, SEQ ID NO: 14, SEQ ID NO: 15, SEQ ID NO: 16, or SEQ ID NO: 17, or a sequence having 80%, 85%, 86%, 87%, 88%, 89%, 90%, 91%, 92%, 93%, 94%, 95%, 96%, 97%, 98%, or 99% sequence identity to SEQ ID NO: 12, SEQ ID NO: 13, SEQ ID NO: 14, SEQ ID NO: 15, SEQ ID NO: 16, or SEQ ID NO: 17. In certain embodiments, in any of the foregoing adenoviruses, the recombinant adenovirus comprises the nucleotide sequence of SEQ ID NO: 18 or SEQ ID NO: 19, or a sequence having 80%, 85%, 86%, 87%, 88%, 89%, 90%, 91%, 92%, 93%, 94%, 95%, 96%, 97%, 98%, or 99% sequence identity to SEQ ID NO: 18 or SEQ ID NO: 19.

In certain embodiments, in any of the foregoing adenoviruses, the recombinant adenovirus comprises the nucleotide sequence of SEQ ID NO: 21, or a sequence having 80%, 85%, 86%, 87%, 88%, 89%, 90%, 91%, 92%, 93%, 94%, 95%, 96%, 97%, 98%, or 99% sequence identity to SEQ ID NO: 21.

In certain embodiments, any of the foregoing recombinant adenoviruses may comprise a deletion of at least one Pea3 binding site, or a functional portion thereof, e.g., the adenovirus may comprise a deletion of nucleotides corresponding to about −300 to about −250 upstream of the initiation site of E1a or a deletion of nucleotides corresponding to −304 or −305 to −255 upstream of the initiation site of E1a. In certain embodiments, the recombinant adenovirus may comprise a deletion of nucleotides corresponding to 195-244 of the Ad5 genome (SEQ ID NO: 1), and/or the recombinant adenovirus may comprise the sequence GGTGTTTTGG (SEQ ID NO: 22). In certain embodiments, any of the foregoing recombinant adenoviruses may comprise a deletion of at least one Pea3 binding site, or a functional portion thereof, and not comprise a deletion of an E2F binding site.

In certain embodiments, any of the foregoing recombinant adenoviruses may comprise a deletion of at least one E2F binding site, or a functional portion thereof. In certain embodiments, any of the foregoing recombinant adenoviruses may comprise a deletion of at least one E2F binding site, or a functional portion thereof, and not comprise a deletion of a Pea3 binding site.

In certain embodiments, any of the foregoing recombinant adenoviruses may comprise an E1a promoter having a deletion of a functional TATA box, e.g., the deletion of an entire TATA box. For example, in certain embodiments, the adenovirus comprises a deletion of nucleotides corresponding to −27 to −24, −31 to −24, −44 to +54, or −146 to +54 of the adenovirus type 5 E1a promoter, which correspond, respectively, to nucleotides 472 to 475, 468 to 475, 455 to 552, and 353 to 552 of the Ad5 genome (SEQ ID NO: 1). In certain embodiments, the adenovirus may comprise a deletion of nucleotides corresponding to −29 to −26, −33 to −26, −44 to +52, or −148 to +52 of the E1a promoter. In certain embodiments, the adenovirus comprises a polynucleotide deletion that results in an adenovirus comprising the sequence CTAGGACTG (SEQ ID NO: 23), AGTGCCCG (SEQ ID NO: 30), or TATTCCCG (SEQ ID NO: 31), which result from joining the two polynucleotide sequences that would otherwise flank the deleted polynucleotide sequence. In certain embodiments, the deletion comprises a deletion of nucleotides corresponding to 353-552 of the Ad5 genome (SEQ ID NO: 1), and/or the E1a promoter comprises the sequence CTAGGACTG (SEQ ID NO: 23).

In certain embodiments, any of the foregoing recombinant adenoviruses may comprise an E1a promoter having a deletion of a functional CAAT box, e.g., the deletion of an entire CAAT box. For example, in certain embodiments, the adenovirus comprises a deletion of nucleotides corresponding to −76 to −68 of the adenovirus type 5 E1a promoter, which corresponds to nucleotides 423 to 431 of the Ad5 genome (SEQ ID NO: 1). In certain embodiments, the adenovirus comprises a polynucleotide deletion that results in an adenovirus comprising the sequence TTCCGTGGCG (SEQ ID NO: 32), which results from joining the two polynucleotide sequences that would otherwise flank the deleted polynucleotide sequence.

In certain embodiments, the first and/or second therapeutic transgenes are not operably linked to an exogenous promoter sequence. In certain embodiments, neither of the therapeutic transgenes are operably linked to an exogenous promoter sequence.

In certain embodiments, any of the foregoing recombinant adenoviruses may selectively replicate in a hyperproliferative cell. In certain embodiments, any of the foregoing recombinant adenoviruses may selectively express endostatin and/or angiostatin in a hyperproliferative cell. The hyperproliferative cell may be a cancer cell, e.g., a lung cancer cell, a colon cancer cell, and a pancreatic cancer cell. In certain embodiments, any of the foregoing recombinant adenoviruses may be an oncolytic adenovirus.

In another aspect, the invention provides a pharmaceutical composition comprising any of the foregoing recombinant adenoviruses and at least one pharmaceutically acceptable carrier or diluent.

In another aspect, the invention provides a method of treating cancer in a subject. The method comprises administering to the subject an effective amount of a combination of (i) a recombinant adenovirus and (ii) an anti-angiogenic agent to treat the cancer in the subject.

In certain embodiments, the anti-angiogenic agent is selected from aflibercept, an anti-VEGF antibody (e.g., bevacizumab and ranibizumab), sunitinib, pazopanib, sorafenib, regorafenib, vandetanib, cabozantinib, axitinib, tivozanib, linifanib, pegaptanib, spironolactone, indomethacin, thalidomide, interleukin-12, an anti-FGF antibody, a tyrosine kinase inhibitor, an interferon, suramin, a suramin analog, somatostatin, and a somatostatin analog. In certain embodiments, the anti-angiogenic agent is selected from aflibercept, bevacizumab, ranibizumab, sunitinib, pazopanib, sorafenib, regorafenib, vandetanib, cabozantinib, axitinib, tivozanib and linifanib. In certain embodiments, the anti-angiogenic agent is bevacizumab, e.g., bevacizumab administered as a dose of from about 1 mg/kg to about 5 mg/kg, or bevacizumab administered at a dose of about 2.5 mg/kg.

In certain embodiments of any of the foregoing methods, the recombinant adenovirus may comprise a deletion of at least one Pea3 binding site, or a functional portion thereof, e.g., the adenovirus may comprise a deletion of nucleotides corresponding to about −300 to about −250 upstream of the initiation site of E1a or a deletion of nucleotides corresponding to −304 to −255 upstream of the initiation site of E1a. In certain embodiments, the recombinant adenovirus may comprise a deletion of nucleotides corresponding to 195-244 of the Ad5 genome (SEQ ID NO: 1), and/or the recombinant adenovirus may comprise the sequence GGTGTTTTGG (SEQ ID NO: 22).

In certain embodiments of any of the foregoing methods, the recombinant adenovirus may comprise an E1a promoter having a deletion of a functional TATA box, e.g., the deletion of an entire TATA box. For example, in certain embodiments, the adenovirus comprises a deletion of nucleotides corresponding to −27 to −24, −31 to −24, −44 to +54, or −146 to +54 of the adenovirus type 5 E1a promoter, which correspond, respectively, to nucleotides 472 to 475, 468 to 475, 455 to 552, and 353 to 552 of the Ad5 genome (SEQ ID NO: 1). In certain embodiments, the adenovirus comprises a polynucleotide deletion that results in an adenovirus comprising the sequence CTAGGACTG (SEQ ID NO: 23), AGTGCCCG (SEQ ID NO: 30), or TATTCCCG (SEQ ID NO: 31), which result from joining the two polynucleotide sequences that would otherwise flank the deleted polynucleotide sequence.

In certain embodiments of any of the foregoing methods, the recombinant adenovirus may comprise an E1a promoter having a deletion of a functional CAAT box, e.g., the deletion of an entire CAAT box. For example, in certain embodiments, the adenovirus comprises a deletion of nucleotides corresponding to −76 to −68 of the adenovirus type 5 E1a promoter, which corresponds to nucleotides 423 to 431 of the Ad5 genome (SEQ ID NO: 1). In certain embodiments, the adenovirus comprises a polynucleotide deletion that results in an adenovirus comprising the sequence TTCCGTGGCG (SEQ ID NO: 32), which results from joining the two polynucleotide sequences that would otherwise flank the deleted polynucleotide sequence.

In certain embodiments of any of the foregoing methods, the recombinant adenovirus may selectively replicate in a hyperproliferative cell. In certain embodiments, any of the foregoing recombinant adenoviruses may selectively express endostatin and/or angiostatin in a hyperproliferative cell. The hyperproliferative cell may be a cancer cell, e.g., a lung cancer cell, a colon cancer cell, and a pancreatic cancer cell. In certain embodiments, any of the foregoing recombinant adenoviruses may be an oncolytic adenovirus.

In another aspect, the invention provides a method of treating cancer in a subject. The method comprises administering to the subject an effective amount of a recombinant adenovirus described herein to treat the cancer disease in the subject. The recombinant adenovirus can, e.g., be administered in combination with one or more therapies selected from surgery, radiation, chemotherapy, immunotherapy, hormone therapy, and virotherapy. In certain embodiments, the recombinant adenovirus is administered in combination with an anti-angiogenic agent. In certain embodiments, the anti-angiogenic agent is selected from aflibercept, an anti-VEGF antibody (e.g., bevacizumab and ranibizumab), sunitinib, pazopanib, sorafenib, regorafenib, vandetanib, cabozantinib, axitinib, tivozanib, linifanib, pegaptanib, spironolactone, indomethacin, thalidomide, interleukin-12, an anti-FGF antibody, a tyrosine kinase inhibitor, an interferon, suramin, a suramin analog, somatostatin, and a somatostatin analog. In certain embodiments, the anti-angiogenic agent is selected from aflibercept, bevacizumab, ranibizumab, sunitinib, pazopanib, sorafenib, regorafenib, vandetanib, cabozantinib, axitinib, tivozanib and linifanib. In certain embodiments, the recombinant adenovirus is administered in combination with bevacizumab, e.g., bevacizumab administered as a dose of from about 1 mg/kg to about 5 mg/kg, or bevacizumab administered at a dose of about 2.5 mg/kg.

In certain embodiments of any of the foregoing methods, the cancer is selected from anal cancer, basal cell carcinoma, bladder cancer, bone cancer, brain cancer, breast cancer, carcinoma, cholangiocarcinoma, cervical cancer, colon cancer, colorectal cancer, endometrial cancer, gastroesophageal cancer, gastrointestinal (GI) cancer, gastrointestinal stromal tumor, hepatocellular carcinoma, gynecologic cancer, head and neck cancer, hematologic cancer, kidney cancer, leukemia, liver cancer, lung cancer, lymphoma, melanoma, merkel cell carcinoma, mesothelioma, neuroendocrine cancer, non-small cell lung cancer, ovarian cancer, pancreatic cancer, pediatric cancer, prostate cancer, renal cell carcinoma, sarcoma, skin cancer, small cell lung cancer, squamous cell carcinoma of the skin, stomach cancer, testicular cancer and thyroid cancer.

In certain embodiments of any of the foregoing methods, the cancer is selected from gastroesophageal cancer (e.g., gastric or gastro-esophageal junction adenocarcinoma), non-small cell lung cancer (e.g., metastatic NSCLC), colorectal cancer (e.g., metastatic colorectal cancer), ovarian cancer (e.g., platinum-resistant ovarian cancer), leukemia, cervical cancer (e.g., late-stage cervical cancer) brain and central nervous system cancer (e.g., glioblastoma), kidney cancer (e.g., renal cell carcinoma), a sarcoma (e.g., rhabdomyosarcoma, osteosarcoma, and Ewing sarcoma), lymphoma (e.g., Hodgkin and non-Hodgkin), ocular cancer (e.g., choroidal melanoma and retinoblastoma), and von Hippel-Lindau disease.

In certain embodiments of any of the foregoing methods, the cancer is selected from brain and central nervous system cancer (e.g., astrocytoma, brain stem glioma, craniopharyngioma, desmoplastic infantile ganglioglioma, ependymoma, high-grade glioma, medulloblastoma, atypical teratoid rhabdoid tumor, neuroblastoma), kidney cancer (e.g., Wilms tumor), ocular cancer (e.g., retinoblastoma), a sarcoma (e.g., rhabdomyosarcoma, osteosarcoma, and Ewing sarcoma), liver cancer (e.g., hepatoblastoma and hepatocellular carcinoma), lymphoma (e.g., Hodgkin and non-Hodgkin), leukemia, and a germ cell tumor.

In another aspect, the invention provides a method of inhibiting proliferation of a tumor cell in a subject. The method comprises administering to the subject an effective amount of a recombinant adenovirus described herein to inhibit proliferation of the tumor cell.

In another aspect, the invention provides a method of inhibiting tumor growth in a subject. The method comprises administering to the subject an effective amount of a recombinant adenovirus described herein to inhibit proliferation of the tumor cell.

In certain embodiments of any of the foregoing methods, the recombinant adenovirus is administered in combination with a second recombinant adenovirus. In certain embodiments, the second recombinant adenovirus is an oncolytic adenovirus. In certain embodiments, the second recombinant adenovirus comprises a nucleotide sequence encoding a polypeptide, or a fragment thereof, selected from acetylcholine, an androgen-receptor, an anti-PD-1 antibody heavy chain and/or light chain, an anti-PD-L1 antibody heavy chain and/or light chain, BORIS/CTCFL, BRAF, CD19, CD20, CD30, CD80, CD86, CD137, CD137L, CD154, CEA, DKK1/Wnt, EGFRvIII, FGF, gp100, Her-2/neu, ICAM, IL-1, IL-3, IL-4, IL-5, IL-6, IL-8, IL-9, IL-17, IL-23A/p19, p40, IL-24, IL-27, IL-27A/p28, IL-27B/EBI3, IL-35, interferon-gamma, KRAS, MAGE, MAGE-A3, MART1, melan-A, mesothelin, MUC-1, NY-ESO-1, Podocalyxin (Podxl), p53, TGF-β, a TGF-β trap, thymidine kinase, and tyrosinase. In certain embodiments, the second recombinant adenovirus comprises a nucleotide sequence encoding a cancer antigen derived from 9D7, androgen receptor, a BAGE family protein, β-catenin, BING-4, BRAF, BRCA1/2, a CAGE family protein, calcium-activated chloride channel 2, CD19, CD20, CD30, CDK4, CEA, CML66, CT9, CT10, cyclin-B1, EGFRvIII, Ep-CAM, EphA3, fibronectin, a GAGE family protein, gp100/pme117, Her-2/neu, HPV E6, HPV E7, Ig, immature laminin receptor, a MAGE family protein (e.g., MAGE-A3), MART-1/melan-A, MART2, MC1R, mesothelin, a mucin family protein (e.g., MUC-1), NY-ESO-1/LAGE-1, P.polypeptide, p53, podocalyxin (Podxl), PRAME, a ras family proteins (e.g., KRAS), prostate specific antigen, a SAGE family protein, SAP-1, SSX-2, survivin, TAG-72, TCR, telomerase, TGF-βRII, TRP-1, TRP-2, tyrosinase, or a XAGE family protein.

In another aspect, the invention provides a method of lowering blood pressure in a subject in need thereof. The method comprises administering to the subject an effective amount of a recombinant adenovirus described herein to lower blood pressure in the subject. In another aspect, the invention provides a method of increasing nitric oxide (NO) production in a subject in need thereof. The method comprises administering to the subject an effective amount of a recombinant adenovirus described herein to increase nitric oxide (NO) production in the subject. In another aspect, the invention provides a method of treating and/or preventing hypertension in a subject in need thereof. The method comprises administering to the subject an effective amount of a recombinant adenovirus described herein to treat and/or prevent hypertension in the subject. In each of the foregoing aspects, the subject may also be receiving or have received a VEGF inhibitor.

In each of the foregoing methods, the effective amount of the recombinant adenovirus can be, e.g., 10²-10¹⁵ plaque forming units (pfus). In each of the foregoing methods, the subject can, e.g., be a human, e.g., a pediatric human, or an animal.

In each of the foregoing methods, the recombinant adenovirus can be, e.g., administered to the subject by oral, parenteral, transdermal, topical, intravenous, subcutaneous, intramuscular, intradermal, ophthalmic, epidural, intratracheal, sublingual, buccal, rectal, vaginal, nasal or inhalation administration.

In another aspect, the invention provides a method of expressing endostatin and/or angiostatin in a target cell. The method comprises exposing the cell to an effective amount of the recombinant adenovirus described herein to express the target transgenes.

These and other aspects and advantages of the invention are illustrated by the following figures, detailed description and claims.

### DESCRIPTION OF THE DRAWINGS

The invention can be more completely understood with reference to the following drawings.

FIGS. 1A-1H are line graphs showing the anti-tumor effects of endostatin or angiostatin expressing oncolytic adenoviruses and/or an anti-VEGF-A antibody in mice carrying subcutaneous ADS-12 tumors, where FIG. 1A represents treatment with a phosphate buffered saline (“PBS”) and a viral formulation buffer (“Buffer”) control, FIG. 1B represents treatment with a mouse ortholog of bevacizumab (“Bev”) and a viral formulation buffer control (“Buffer”), FIG. 1C represents treatment with the angiostatin expressing TAV-Ang adenovirus (“Ang”) and a phosphate buffered saline control (“PBS”), FIG. 1D represents combination therapy with a mouse ortholog of bevacizumab (“Bev”) and the angiostatin expressing TAV-Ang adenovirus (“Ang”), FIG. 1E represents treatment with the endostatin expressing TAV-Endo adenovirus (“Endo”) and a phosphate buffered saline control (“PBS”), FIG. 1F represents a combination therapy with a mouse ortholog of bevacizumab (“Bev”) and the endostatin expressing TAV-Endo adenovirus (“Endo”), FIG. 1G represents treatment with the empty TAV-Δ19k adenovirus (“19k”) and a phosphate buffered saline control (“PBS”), and FIG. 1H represents a combination therapy with a mouse ortholog of bevacizumab (“Bev”) and the empty TAV-Δ19k adenovirus (“19k”). TAV-Ang, TAV-Endo, TAV-Δ19k, and viral formulation buffer were administered by intratumoral injection on days 0, 4, and 8, and PBS and Bev were administered by intraperitoneal injection on days 1, 5, 7, and 9. Each line represents the tumor volume of one mouse (n=10 per group). Tumor volumes were estimated as length·width²/2.

FIG. 2 is line graph depicting the mean of the individual tumor volumes shown in FIG. 1.

FIG. 3 is line graph showing progression free survival for the treatment groups depicted in FIG. 1.

FIG. 4 depicts results obtained from tracking the same treatment groups described in FIG. 1 for a longer period of time. FIGS. 4A-4H are line graphs showing the anti-tumor effects of endostatin or angiostatin expressing oncolytic adenoviruses and/or an anti-VEGF-A antibody in mice carrying subcutaneous ADS-12 tumors, where FIG. 4A represents treatment with a phosphate buffered saline (“PBS”) and a viral formulation buffer (“Buffer”) control, FIG. 4B represents treatment with a mouse ortholog of bevacizumab (“Bev”) and a viral formulation buffer control (“Buffer”), FIG. 4C represents treatment with the angiostatin expressing TAV-Ang adenovirus (“Ang”) and a phosphate buffered saline control (“PBS”), FIG. 4D represents combination therapy with a mouse ortholog of bevacizumab (“Bev”) and the angiostatin expressing TAV-Ang adenovirus (“Ang”), FIG. 4E represents treatment with the endostatin expressing TAV-Endo adenovirus (“Endo”) and a phosphate buffered saline control (“PBS”), FIG. 4F represents a combination therapy with a mouse ortholog of bevacizumab (“Bev”) and the endostatin expressing TAV-Endo adenovirus (“Endo”), FIG. 4G represents treatment with the empty TAV-Δ19k adenovirus (“19k”) and a phosphate buffered saline control (“PBS”), and FIG. 4H represents a combination therapy with a mouse ortholog of bevacizumab (“Bev”) and the empty TAV-Δ19k adenovirus (“19k”). TAV-Ang, TAV-Endo, TAV-Δ19k, and viral formulation buffer were administered by intratumoral injection on days 0, 4, and 8, and PBS and Bev were administered by intraperitoneal injection on days 1, 5, 7, and 9. Each line represents the tumor volume of one mouse (n=10 per group). Tumor volumes were estimated as length·width²/2. FIG. 4 and FIG. 1 represent data from the same set of experiments.

FIG. 5 is line graph depicting the mean of the individual tumor volumes shown in FIG. 4.

FIG. 6 is line graph showing progression free survival for the treatment groups depicted in FIG. 4.

FIG. 7 shows line graphs depicting primary tumor volume (top) and secondary tumor volume (bottom) in mice treated with angiostatin expressing oncolytic adenoviruses as described in Example 4.

FIGS. 8A-8D are line graphs showing the anti-tumor effects of an oncolytic adenovirus and/or an anti-VEGF-A antibody in mice carrying subcutaneous ADS-12 tumors, where FIG. 8A represents treatment with a phosphate buffered saline (“PBS”) and a viral formulation buffer (“Buffer”) control, FIG. 8B represents treatment with a mouse ortholog of bevacizumab (“Bev”) and a viral formulation buffer control (“Buffer”), FIG. 8C represents treatment with the empty TAV-Δ19k adenovirus (“19k”) and a phosphate buffered saline control (“PBS”), FIG. 8D represents a combination therapy with a mouse ortholog of bevacizumab (“Bev”) and the empty TAV-Δ19k adenovirus (“19k”). TAV-Δ19k and viral formulation buffer were administered by intratumoral injection on days 0, 4, and 8, and PBS and Bev were administered by intraperitoneal injection on days 1, 5, 7, and 9. Each line represents the tumor volume of one mouse. Tumor volumes were estimated as length·width2/2.

FIG. 9 is a table showing the cure rate (complete tumor remission) for the treatment groups depicted in FIG. 8.

### DETAILED DESCRIPTION

The invention is based, in part, upon the discovery of recombinant adenoviruses that can efficiently express anti-angiogenic factors such as endostatin and/or angiostatin. Additionally, the invention is based, in part, upon the discovery that an anti-cancer treatment using an anti-VEGF antibody, e.g., bevacizumab, can be enhanced when the anti-VEGF antibody is administered in combination with a recombinant adenovirus, e.g., an endostatin and/or angiostatin expressing adenovirus described herein. Surprisingly, it has been discovered that for certain cancers, the recombinant adenoviruses described herein, administered alone or in combination with an anti-VEGF antibody, e.g., bevacizumab, do not merely slow or stop cancer growth but cause a cancer to go in to partial and/or complete remission.

Accordingly, in one aspect, the invention provides a recombinant adenovirus comprising a first nucleotide sequence encoding a first therapeutic transgene selected from endostatin and angiostatin inserted into an E1b-19K insertion site; wherein the E1b-19K insertion site is located between the start site of E1b-19K (i.e., the nucleotide sequence encoding the start codon of E1b-19k, e.g., corresponding to nucleotides 1714-1716 of SEQ ID NO: 1) and the start site of E1b-55K (i.e., the nucleotide sequence encoding the start codon of E1b-55k, e.g., corresponding to nucleotides 2019-2021 of SEQ ID NO: 1). Throughout the description and claims, an insertion between two sites, for example, an insertion between (i) a start site of a first gene (e.g., E1b-19k) and a start site of a second gene, (e.g., E1b-55K), (ii) a start site of a first gene and a stop site of a second gene, (iii) a stop site of a first gene and start site of a second gene, or (iv) a stop site of first gene and a stop site of a second gene, is understood to mean that all or a portion of the nucleotides constituting a given start site or a stop site surrounding the insertion may be present or absent in the final virus. Similarly, an insertion between two nucleotides is understood to mean that the nucleotides surrounding the insertion may be present or absent in the final virus. The term “transgene” refers to an exogenous gene or polynucleotide sequence. The term “therapeutic transgene” refers to a transgene, which when replicated and/or expressed in or by the virus imparts a therapeutic effect in a target cell, body fluid, tissue, organ, physiological system, or subject.

In certain embodiments, the E1b-19K insertion site is located between the start site of E1b-19K (i.e., the nucleotide sequence encoding the start codon of E1b-19k, e.g., corresponding to nucleotides 1714-1716 of SEQ ID NO: 1) and the stop site of E1b-19K (i.e., the nucleotide sequence encoding the stop codon of E1b-19k, e.g., corresponding to nucleotides 2242-2244 of SEQ ID NO: 1). In certain embodiments, the E1b-19K insertion site comprises a deletion of from about 100 to about 305, about 100 to about 300, about 100 to about 250, about 100 to about 200, about 100 to about 150, about 150 to about 305, about 150 to about 300, about 150 to about 250, or about 150 to about 200 nucleotides adjacent the start site of E1b-19K. In certain embodiments, the E1b-19K insertion site comprises a deletion of about 200 nucleotides, e.g., 202 or 203 nucleotides adjacent the start site of E1b-19K. In certain embodiments, the E1b-19K insertion site comprises a deletion corresponding to nucleotides 1714-1916 of the Ad5 genome (SEQ ID NO: 1). In certain embodiments, the first therapeutic transgene is inserted between nucleotides corresponding to 1713 and 1917 of the Ad5 genome (SEQ ID NO: 1). In certain embodiments, the first therapeutic transgene is inserted between CTGACCTC (SEQ ID NO: 2) and TCACCAGG (SEQ ID NO: 3), e.g., the recombinant adenovirus comprises, in a 5′ to 3′ orientation, CTGACCTC (SEQ ID NO: 2), the first therapeutic transgene, and TCACCAGG (SEQ ID NO: 3). CTGACCTC (SEQ ID NO: 2) and TCACCAGG (SEQ ID NO: 3) define unique boundary sequences for the E1b-19K insertion site within the Ad5 genome (SEQ ID NO: 1). Throughout the description and claims, a deletion adjacent to a site, for example, a deletion adjacent to a start site of a gene or a deletion adjacent to a stop site of a gene, is understood to mean that the deletion may include a deletion of all, a portion, or none of the nucleotides constituting a given start site or a stop site.

In certain embodiments, the recombinant adenovirus comprises a second nucleotide sequence encoding a second therapeutic transgene selected from endostatin and angiostatin, wherein the second therapeutic transgene is inserted into the E1b-19k insertion site, and the first nucleotide sequence and the second nucleotide sequence are separated by an internal ribosome entry site (IRES). The IRES may, e.g., be selected from an encephalomyocarditis virus (EMCV) IRES, a foot-and-mouth disease virus (FMDV) IRES, and a poliovirus IRES. The IRES may, e.g., comprise SEQ ID NO: 20. In certain embodiments, the first and second therapeutic transgenes are inserted between nucleotides corresponding to 1713 and 1917 of the Ad5 genome (SEQ ID NO: 1), e.g., the first and second therapeutic transgenes are inserted between CTGACCTC (SEQ ID NO: 2) and TCACCAGG (SEQ ID NO: 3), e.g., the recombinant adenovirus comprises, in a 5′ to 3′ orientation, CTGACCTC (SEQ ID NO: 2), the first therapeutic transgene, the IRES, the second therapeutic transgene, and TCACCAGG (SEQ ID NO: 3).

In certain embodiments the recombinant adenovirus comprises an E3 deletion. In certain embodiments, the E3 deletion comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or from about 3000 to about 3185 nucleotides. In certain embodiments the E3 deletion is located between the stop site of pVIII (i.e., the nucleotide sequence encoding the stop codon of pVIII, e.g., corresponding to nucleotides 27855-27857 of SEQ ID NO: 1) and the start site of Fiber (i.e., the nucleotide sequence encoding the start codon of Fiber, e.g., corresponding to nucleotides 31042-31044 of SEQ ID NO: 1). In certain embodiments, the E3 deletion site is located between the stop site of E3-10.5K (i.e., the nucleotide sequence encoding the stop codon of E3-10.5K, e.g., corresponding to nucleotides 29770-29772 of SEQ ID NO: 1) and the stop site of E3-14.7K (i.e., the nucleotide sequence encoding the stop codon of E3-14.7K, e.g., corresponding to nucleotides 30837-30839 of SEQ ID NO: 1). In certain embodiments, the E3 deletion comprises a deletion of from about 500 to about 1551, from about 500 to about 1500, from about 500 to about 1000, from about 1000 to about 1551, from about 1000 to about 1500, or from about 1500 to about 1551 nucleotides adjacent the stop site of E3-10.5K. In certain embodiments, the E3 deletion comprises a deletion of about 1050 nucleotides adjacent the stop site of E3-10.5K, e.g., the E3 deletion comprises a deletion of 1063 or 1064 nucleotides adjacent the stop site of E3-10.5K. In certain embodiments, the E3 deletion comprises a deletion corresponding to the Ad5 dl309 E3 deletion. In certain embodiments, the E3 deletion comprises a deletion corresponding to nucleotides 29773-30836 of the Ad5 genome (SEQ ID NO: 1).

In certain embodiments, the E3 deletion is located between stop site of E3-gp19K (i.e., the nucleotide sequence encoding the stop codon of E3-gp19K, e.g., corresponding to nucleotides 29215-29217 of SEQ ID NO: 1) and the stop site of E3-14.7K (i.e., the nucleotide sequence encoding the stop codon of E3-14.7K, e.g., corresponding to nucleotides 30837-30839 of SEQ ID NO: 1). In certain embodiments, the E3 deletion comprises a deletion of from about 500 to about 1824, from about 500 to about 1500, from about 500 to about 1000, from about 1000 to about 1824, from about 1000 to about 1500, or from about 1500 to about 1824 nucleotides adjacent the stop site of E3-gp19K. In certain embodiments, the E3 deletion comprises a deletion of about 1600 nucleotides adjacent the stop site of E3-gp19K. e.g., the E3 deletion comprises a deletion of 1622 nucleotides adjacent the stop site of E3-gp19K. In certain embodiments, the E3 deletion comprises a deletion corresponding to nucleotides 29218-30839 of the Ad5 genome (SEQ ID NO: 1).

In certain embodiments, the recombinant adenovirus comprises a second nucleotide sequence encoding a second therapeutic transgene selected from endostatin and angiostatin, wherein the second therapeutic transgene is inserted into an E3 insertion site. In certain embodiments, the E3 insertion site is located between the stop site of pVIII (i.e., the nucleotide sequence encoding the stop codon of pVIII, e.g., corresponding to nucleotides 27855-27857 of SEQ ID NO: 1) and the start site of Fiber (i.e., the nucleotide sequence encoding the start codon of Fiber, e.g., corresponding to nucleotides 31042-31044 of SEQ ID NO: 1). In certain embodiments, the E3 insertion site comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or from about 3000 to about 3185 nucleotides. In certain embodiments, the E3 insertion site is located between the stop site of E3-10.5K (i.e., the nucleotide sequence encoding the stop codon of E3-10.5K, e.g., corresponding to nucleotides 29770-29772 of SEQ ID NO: 1) and the stop site of E3-14.7K (i.e., the nucleotide sequence encoding the stop codon of E3-14.7K, e.g., corresponding to nucleotides 30837-30839 of SEQ ID NO: 1). In certain embodiments, the E3 insertion site comprises a deletion of from about 500 to about 1551, from about 500 to about 1500, from about 500 to about 1000, from about 1000 to about 1551, from about 1000 to about 1500, or from about 1500 to about 1551 nucleotides adjacent the stop site of E3-10.5K. In certain embodiments, the E3 insertion site comprises a deletion of about 1050 nucleotides adjacent the stop site of E3-10.5K, e.g., the E3 insertion site comprises a deletion of 1063 or 1064 nucleotides adjacent the stop site of E3-10.5K. In certain embodiments, the E3 insertion site comprises a deletion corresponding to the Ad5 dl309 E3 deletion. In certain embodiments, the E3 insertion site comprises a deletion corresponding to nucleotides 29773-30836 of the Ad5 genome (SEQ ID NO: 1). In certain embodiments, the second therapeutic transgene is inserted between nucleotides corresponding to 29773 and 30836 of the Ad5 genome (SEQ ID NO: 1). In certain embodiments, the second therapeutic transgene is inserted between CAGTATGA (SEQ ID NO: 4) and TAATAAAAAA (SEQ ID NO: 5), e.g., the recombinant adenovirus comprises, in a 5′ to 3′ orientation, CAGTATGA (SEQ ID NO: 4), the second therapeutic transgene, and TAATAAAAAA (SEQ ID NO: 5). CAGTATGA (SEQ ID NO: 4) and TAATAAAAAA (SEQ ID NO: 5) define unique boundary sequences for an E3 insertion site within the Ad5 genome (SEQ ID NO: 1).

In certain embodiments, the E3 insertion site is located between stop site of E3-gp19K (i.e., the nucleotide sequence encoding the stop codon of E3-gp19K, e.g., corresponding to nucleotides 29215-29217 of SEQ ID NO: 1) and the stop site of E3-14.7K (i.e., the nucleotide sequence encoding the stop codon of E3-14.7K, e.g., corresponding to nucleotides 30837-30839 of SEQ ID NO: 1). In certain embodiments, the E3 insertion site comprises a deletion of from about 500 to about 1824, from about 500 to about 1500, from about 500 to about 1000, from about 1000 to about 1824, from about 1000 to about 1500, or from about 1500 to about 1824 nucleotides adjacent the stop site of E3-gp19K. In certain embodiments, the E3 insertion site comprises a deletion of about 1600 nucleotides adjacent the stop site of E3-gp19K. e.g., the E3 insertion site comprises a deletion of 1622 nucleotides adjacent the stop site of E3-gp19K. In certain embodiments, the E3 insertion site comprises a deletion corresponding to nucleotides 29218-30839 of the Ad5 genome (SEQ ID NO: 1). In certain embodiments, the second therapeutic transgene is inserted between nucleotides corresponding to 29218 and 30839 of the Ad5 genome (SEQ ID NO: 1). In certain embodiments, the second therapeutic transgene is inserted between TGCCTTAA (SEQ ID NO: 33) and TAAAAAAAAAT (SEQ ID NO: 34), e.g., the recombinant adenovirus comprises, in a 5′ to 3′ orientation, TGCCTTAA (SEQ ID NO: 33), the second therapeutic transgene, and TAAAAAAAAAT (SEQ ID NO: 34). TGCCTTAA (SEQ ID NO: 33) and TAAAAAAAAAT (SEQ ID NO: 34) define unique boundary sequences for an E3 insertion site within the Ad5 genome (SEQ ID NO: 1).

In certain embodiments, the recombinant adenovirus comprises an E4 deletion. In certain embodiments, the E4 deletion is located between the start site of E4-ORF6/7 (i.e., the nucleotide sequence encoding the start codon of E4-ORF6/7, e.g., corresponding to nucleotides 34075-34077 of SEQ ID NO: 1) and the right inverted terminal repeat (ITR; e.g., corresponding to nucleotides 35836-35938 of SEQ ID NO: 1). In certain embodiments, the E4 deletion is located between the start site of E4-ORF6/7 and the start site of E4-ORF1 (i.e., the nucleotide sequence encoding the start codon of E4-ORF1, e.g., corresponding to nucleotides 35524-35526 of SEQ ID NO: 1). In certain embodiments, the E4 deletion comprises a deletion of a nucleotide sequence between the start site of E4-ORF6/7 and the start site of E4-ORF1. In certain embodiments, the E4 deletion comprises a deletion of from about 500 to about 2500, from about 500 to about 2000, from about 500 to about 1500, from about 500 to about 1000, from about 1000 to about 2500, from about 1000 to about 2000, from about 1000 to about 1500, from about 1500 to about 2500, from about 1500 to about 2000, or from about 2000 to about 2500 nucleotides. In certain embodiments, the E4 deletion comprises a deletion of from about 250 to about 1500, from about 250 to about 1250, from about 250 to about 1000, from about 250 to about 750, from about 250 to about 500, from 500 to about 1500, from about 500 to about 1250, from about 500 to about 1000, from about 500 to about 750, from 750 to about 1500, from about 750 to about 1250, from about 750 to about 1000, from about 1000 to about 1500, or from about 1000 to about 1250 nucleotides adjacent the start site of E4-ORF6/7. In certain embodiments, the E4 deletion comprises a deletion of about 1450 nucleotides adjacent the start site of E4-ORF6/7, e.g., the E4 deletion comprises a deletion of about 1449 nucleotides adjacent the start site of E4-ORF6/7. In certain embodiments, the E4 deletion comprises a deletion corresponding to nucleotides 34078-35526 of the Ad5 genome (SEQ ID NO: 1).

In certain embodiments, the recombinant adenovirus is an oncolytic adenovirus, e.g., an adenovirus that exhibits tumor-selective replication and/or viral mediated lysis. In certain embodiments, the oncolytic adenovirus allows for selective expression of a therapeutic transgene in a hyperproliferative cell, e.g., a cancer cell, relative to a non-hyperproliferative cell. In certain embodiments, the expression of the therapeutic transgene in a non-hyperproliferative cell is about 90%, about 80%, about 70%, about 60%, about 50%, about 40%, about 30%, about 20%, about 10%, or about 5% of the expression in a hyperproliferative cell. In certain embodiments, the adenovirus exhibits no detectable expression of the therapeutic transgene in a non-hyperproliferative cell. Therapeutic transgene expression may be determined by any appropriate method known in the art, e.g., Western blot or ELISA.

The hyperproliferative cell may be a cancer cell, e.g., a carcinoma, sarcoma, leukemia, lymphoma, prostate cancer, lung cancer, gastrointestinal tract cancer, colorectal cancer, pancreatic cancer, breast cancer, ovarian cancer, cervical cancer, stomach cancer, thyroid cancer, mesothelioma, liver cancer, kidney cancer, skin cancer, head and neck cancer, or brain cancer cell, which are discussed in more detail below in Section IV.

### I. Viruses

The term “virus” is used herein to refer any of the obligate intracellular parasites having no protein-synthesizing or energy-generating mechanism. The viral genome may be RNA or DNA. The viruses useful in the practice of the present invention include recombinantly modified enveloped or non-enveloped DNA and RNA viruses, preferably selected from baculoviridiae, parvoviridiae, picornoviridiae, herpesviridiae, poxyiridae, or adenoviridiae. A recombinantly modified virus is referred to herein as a “recombinant virus.” A recombinant virus may, e.g., be modified by recombinant DNA techniques to be replication deficient, conditionally replicating, or replication competent, and/or be modified by recombinant DNA techniques to include expression of exogenous transgenes. Chimeric viral vectors which exploit advantageous elements of each of the parent vector properties (See, e.g., Feng et al. (1997) NATURE BIOTECHNOLOGY 15:866-870) may also be useful in the practice of the present invention. Although it is generally favored to employ a virus from the species to be treated, in some instances it may be advantageous to use vectors derived from different species that possess favorable pathogenic features. For example, equine herpes virus vectors for human gene therapy are described in PCT Publication No. WO 98/27216. The vectors are described as useful for the treatment of humans as the equine virus is not pathogenic to humans. Similarly, ovine adenoviral vectors may be used in human gene therapy as they are claimed to avoid the antibodies against the human adenoviral vectors. Such vectors are described in PCT Publication No. WO 97/06826.

Preferably, the recombinant virus is an adenovirus. Adenoviruses are medium-sized (90-100 nm), non-enveloped (naked), icosahedral viruses composed of a nucleocapsid and a double-stranded linear DNA genome. Adenoviruses replicate in the nucleus of mammalian cells using the host's replication machinery. The term “adenovirus” refers to any virus in the genus Adenoviridiae including, but not limited to, human, bovine, ovine, equine, canine, porcine, murine, and simian adenovirus subgenera. In particular, human adenoviruses includes the A-F subgenera as well as the individual serotypes thereof, the individual serotypes and A-F subgenera including but not limited to human adenovirus types 1, 2, 3, 4, 4a, 5, 6, 7, 8, 9, 10, 11 (Ad11a and Ad11p), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91. Preferred are recombinant viruses derived from human adenovirus types 2 and 5. Unless stated otherwise, all adenovirus type 5 nucleotide numbers are relative to the NCBI reference sequence AC_000008.1, which is depicted herein in SEQ ID NO: 1.

The adenovirus replication cycle has two phases: an early phase, during which 4 transcription units (E1, E2, E3, and E4) are expressed, and a late phase which occurs after the onset of viral DNA synthesis, and during which late transcripts are expressed primarily from the major late promoter (MLP). The late messages encode most of the virus's structural proteins. The gene products of E1, E2 and E4 are responsible for transcriptional activation, cell transformation, viral DNA replication, as well as other viral functions, and are necessary for viral growth.

The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

In certain embodiments, the virus has one or more modifications to a regulatory sequence or promoter. A modification to a regulatory sequence or promoter comprises a deletion, substitution, or addition of one or more nucleotides compared to the wild-type sequence of the regulatory sequence or promoter.

In certain embodiments, the modification of a regulatory sequence or promoter comprises a modification of sequence of a transcription factor binding site to reduce affinity for the transcription factor, for example, by deleting a portion thereof, or by inserting a single point mutation into the binding site. In certain embodiments, the additional modified regulatory sequence enhances expression in neoplastic cells, but attenuates expression in normal cells.

In certain embodiments, the modified regulatory sequence is operably linked to a sequence encoding a protein. In certain embodiments, at least one of the adenoviral E1a and E1b genes (coding regions) is operably linked to a modified regulatory sequence. In certain embodiments, the E1a gene is operably linked to the modified regulatory sequence.

The E1a regulatory sequence contains five binding sites for the transcription factor Pea3, designated Pea3 I, Pea3 II, Pea3 III, Pea3 IV, and Pea3 V, where Pea3 I is the Pea3 binding site most proximal to the E1a start site, and Pea3 V is most distal. The E1a regulatory sequence also contains binding sites for the transcription factor E2F, hereby designated E2F I and E2F II, where E2F I is the E2F binding site most proximal to the E1a start site, and E2F II is more distal. From the E1a start site, the binding sites are arranged: Pea3 I, E2F I, Pea3 II, E2F II, Pea3 III, Pea3 IV, and Pea3 V.

In certain embodiments, at least one of these seven binding sites, or a functional portion thereof, is deleted. A “functional portion” is a portion of the binding site that, when deleted, decreases or even eliminates the functionality, e.g. binding affinity, of the binding site to its respective transcription factor (Pea3 or E2F) by, for example, at least 40%, 50%, 60%, 70%, 80%, 90%, 95% or 100% relative to the complete sequence. In certain embodiments, one or more entire binding sites are deleted. In certain embodiments, a functional portion of one or more binding sites is deleted. A “deleted binding site” encompasses both the deletion of an entire binding site and the deletion of a functional portion. When two or more binding sites are deleted, any combination of entire binding site deletion and functional portion deletion may be used.

In certain embodiments, at least one Pea3 binding site, or a functional portion thereof, is deleted. The deleted Pea3 binding site can be Pea3 I, Pea3 II, Pea3 III, Pea3 IV, and/or Pea3 V. In certain embodiments, the deleted Pea3 binding site is Pea3 II, Pea3 III, Pea3 IV, and/or Pea3 V. In certain embodiments, the deleted Pea3 binding site is Pea3 IV and/or Pea3 V. In certain embodiments, the deleted Pea3 binding site is Pea3 II and/or Pea3 III. In certain embodiments, the deleted Pea3 binding site is both Pea3 II and Pea3 III. In certain embodiments, the Pea3 I binding site, or a functional portion thereof, is retained.

In certain embodiments, at least one E2F binding site, or a functional portion thereof, is deleted. In certain embodiments, at least one E2F binding site, or a functional portion thereof, is retained. In certain embodiments, the retained E2F binding site is E2F I and/or E2F II. In certain embodiments, the retained E2F binding site is E2F II. In certain embodiments, the total deletion consists essentially of one or more of Pea3 II, Pea3 III, Pea3 IV, and/or Pea3 V, or functional portions thereof.

In certain embodiments, the recombinant adenovirus has a deletion of a 50 base pair region located from −304 to −255 upstream of the E1a initiation site, e.g., corresponding to 195-244 of the Ad5 genome (SEQ ID NO: 1), hereafter referred to as the TAV-255 deletion. In certain embodiments, the TAV-255 deletion results in an E1a promoter that comprises the sequence GGTGTTTTGG (SEQ ID NO: 22).

In certain embodiments, the recombinant adenovirus comprises an E1a promoter having a deletion of a functional TATA box, e.g., the deletion of an entire TATA box. As used herein, a “functional TATA box” refers to a TATA box that is capable of binding to a TATA box binding protein (TBP), e.g., a TATA box that has at least 100%, at least 90%, at least 80%, at least 70%, at least 60%, at least 50%, or at least 40%, of the TBP binding activity of a corresponding wild-type TATA box sequence. As used herein, a “non-functional TATA box” refers to a TATA box that, e.g., has less than 30%, less than 20%, less than 10%, or 0% of the TBP binding activity of a corresponding wild-type TATA box sequence. Assays for determining whether a TBP binds to a TATA box are known in the art. Exemplary binding assays include electrophoretic mobility shift assays, chromatin immunoprecipitation assays, and DNAse footprinting assays.

For example, in certain embodiments, the recombinant adenovirus comprises a deletion of nucleotides corresponding to −27 to −24, −31 to −24, −44 to +54, or −146 to +54 of the adenovirus type 5 E1a promoter, which correspond, respectively, to nucleotides 472 to 475, 468 to 475, 455 to 552, and 353 to 552 of the Ad5 genome (SEQ ID NO: 1). In certain embodiments, the adenovirus comprises a deletion of nucleotides corresponding to −29 to −26, −33 to −26, −44 to +52, or −148 to +52 of the adenovirus type 5 E1a promoter. In certain embodiments, the adenovirus comprises a deletion of nucleotides corresponding to 353 to 552 of the Ad5 genome (SEQ ID NO: 1). In certain embodiments, the adenovirus comprises a polynucleotide deletion that results in an adenovirus comprising the sequence CTAGGACTG (SEQ ID NO: 23), AGTGCCCG (SEQ ID NO: 30), or TATTCCCG (SEQ ID NO: 31), which result from joining the two polynucleotide sequences that would otherwise flank the deleted polynucleotide sequence. In certain embodiments, the adenovirus comprises a polynucleotide deletion that results in an adenovirus comprising the sequence CTAGGACTG (SEQ ID NO: 23).

In certain embodiments, the recombinant adenovirus comprises an E1a promoter having a deletion of a functional CAAT box, e.g., the deletion of an entire CAAT box. As used herein, a “functional CAAT box” refers to a CAAT box that is capable of binding to a C/EBP or NF-Y protein, e.g., a CAAT box that has at least 100%, at least 90%, at least 80%, at least 70%, at least 60%, at least 50%, or at least 40%, of the a C/EBP or NF-Y binding activity of a corresponding wild-type CAAT box sequence. As used herein, a “non-functional CAAT box” refers to a CAAT box that, e.g., has less than 30%, less than 20%, less than 10%, or 0% of the a C/EBP or NF-Y binding activity of a corresponding wild-type CAAT box sequence. Assays for determining whether a C/EBP or NF-Y protein binds to a CAAT box are known in the art. Exemplary binding assays include electrophoretic mobility shift assays, chromatin immunoprecipitation assays, and DNAse footprinting assays.

For example, in certain embodiments, a recombinant adenovirus comprises a deletion of nucleotides corresponding to −76 to −68 of the adenovirus type 5 E1a promoter, which corresponds to nucleotides 423 to 431 of the Ad5 genome (SEQ ID NO: 1). In certain embodiments, the adenovirus comprises a polynucleotide deletion that results in an adenovirus comprising the sequence TTCCGTGGCG (SEQ ID NO: 32), which results from joining the two polynucleotide sequences that would otherwise flank the deleted polynucleotide sequence.

The adenoviral E1b-19k gene functions primarily as an anti-apoptotic gene and is a homolog of the cellular anti-apoptotic gene, BCL-2. Since host cell death prior to maturation of the progeny viral particles would restrict viral replication, E1b-19k is expressed as part of the E1 cassette to prevent premature cell death thereby allowing the infection to proceed and yield mature virions. Accordingly, in certain embodiments, a recombinant virus is provided that includes an E1b-19K insertion site, e.g., the adenovirus has a nucleotide sequence encoding a therapeutic transgene inserted into an E1b-19K insertion site. In certain embodiments, the adenovirus comprises a nucleotide sequence encoding a therapeutic transgene inserted into an E1b-19K insertion site, wherein the insertion site is located between the start site of E1b-19K (i.e., the nucleotide sequence encoding the start codon of E1b-19k, e.g., corresponding to nucleotides 1714-1716 of SEQ ID NO: 1) and the start site of E1b-55K (i.e., the nucleotide sequence encoding the start codon of E1b-55k, e.g., corresponding to nucleotides 2019-2021 of SEQ ID NO: 1).

In certain embodiments, a recombinant virus is provided that includes an IX-E2 insertion site, e.g., the adenovirus has a nucleotide sequence encoding a therapeutic transgene, e.g., endostatin and/or angiostatin, inserted into an IX-E2 insertion site. In certain embodiments, the IX-E2 insertion site is located between the nucleotide sequence encoding the stop codon of IX and the nucleotide sequence encoding the stop codon of IVa2. In certain embodiments, the nucleotide sequence is inserted between nucleotides corresponding to 4029 and 4093 of the Ad5 genome (SEQ ID NO: 1). In certain embodiments, the nucleotide sequence is inserted between nucleotides corresponding to 4029 and 4050, nucleotides corresponding to 4051 and 4070, or nucleotides corresponding to 4071 and 4093 of the Ad5 genome (SEQ ID NO: 1). In certain embodiments, the IX-E2 insertion site comprises a deletion of about 5, 10, 15, 20, 25, 30, 35, 40, 45, 50, 55, or 60 nucleotides.

In certain embodiments, a recombinant virus is provided that includes an L5-E4 insertion site, e.g., the adenovirus has a nucleotide sequence encoding a therapeutic transgene, e.g., endostatin and/or angiostatin, inserted into an L5-E4 insertion site. In certain embodiments, the L5-E4 insertion site is located between the nucleotide sequence encoding the stop codon of Fiber and the nucleotide sequence encoding the stop codon of E4-ORF6 or E4ORF6/7. In certain embodiments, the nucleotide sequence is inserted between nucleotides corresponding to 32785 to 32916 of the Ad5 genome (SEQ ID NO: 1). In certain embodiments, the nucleotide sequence is inserted between nucleotides corresponding to 32785 and 32800, nucleotides corresponding to 32801 and 32820, nucleotides corresponding to 32821 and 32840, nucleotides corresponding to 32841 and 32860, nucleotides corresponding to 32861 and 32880, nucleotides corresponding to 32881 and 32900, or nucleotides corresponding to 32901 and 32916 of the Ad5 genome (SEQ ID NO: 1). In certain embodiments, the L5-E4 insertion site comprises a deletion of about 5, 10, 15, 20, 25, 30, 35, 40, 45, 50, 55, 60, 65, 70, 75, 80, 85, 90, 95, 100, 105, 110, 115, 120, 125, or 130 nucleotides.

### II. Methods of Viral Production

Methods for producing recombinant viruses of the invention are known in the art. Typically, a disclosed virus is produced in a suitable host cell line using conventional techniques including culturing a transfected or infected host cell under suitable conditions so as to allow the production of infectious viral particles. Nucleic acids encoding viral genes can be incorporated into plasmids and introduced into host cells through conventional transfection or transformation techniques. Exemplary suitable host cells for production of disclosed viruses include human cell lines such as HeLa, Hela-S3, HEK293, 911, A549, HER96, or PER-C6 cells. Specific production and purification conditions will vary depending upon the virus and the production system employed. For adenovirus, the traditional method for the generation of viral particles is co-transfection followed by subsequent in vivo recombination of a shuttle plasmid (usually containing a small subset of the adenoviral genome and optionally containing a potential transgene an expression cassette) and an adenoviral helper plasmid (containing most of the entire adenoviral genome).

Alternative technologies for the generation of adenovirus include utilization of the bacterial artificial chromosome (BAC) system, in vivo bacterial recombination in a recA+bacterial strain utilizing two plasmids containing complementary adenoviral sequences, and the yeast artificial chromosome (YAC) system.

Following production, infectious viral particles are recovered from the culture and optionally purified. Typical purification steps may include plaque purification, centrifugation, e.g., cesium chloride gradient centrifugation, clarification, enzymatic treatment, e.g., benzonase or protease treatment, chromatographic steps, e.g., ion exchange chromatography or filtration steps.

### III. Therapeutic Transgenes

A disclosed recombinant virus may comprise a nucleotide sequence that encodes for a therapeutic transgene selected from endostatin and angiostatin. In certain embodiments, a disclosed recombinant comprise virus may comprise a first nucleotide sequence and a second nucleotide sequence that encode for a first and a second therapeutic transgene, respectively. The first and/or second therapeutic transgene may be selected from endostatin and angiostatin.

When tumors grow beyond approximately 2 mm³ in diameter, they require the proliferation of an independent network of blood vessels to supply nutrients and oxygen and remove waste products. This new vessel formation, i.e., neovascularization, is known as tumor angiogenesis. Pro-angiogenic factors include vascular endothelial growth factor (VEGF), basic fibroblast growth factor (bFGF), platelet-derived growth factor (PDGF), epidermal growth factor (EGF), interleukin 8 (IL-8), and the angiopoietins. Endostatin and angiostatin are naturally occurring anti-angiogenic proteins that are reported to inhibit neovascularization.

Endostatin is a proteolytic fragment of collagen XVIII. An exemplary human collagen XVIII amino acid sequence, corresponding to NCBI Reference Sequence NP_085059.2, is depicted in SEQ ID NO: 6.

Endostatin can result from proteolytic cleavage of collagen XVIII at different sites. The non-collagenous 1 (NC1) domain at the C-terminus of collagen XVIII is generally considered responsible for the anti-angiogenic effects of endostatin. An exemplary human collagen XVIII NC1 domain amino acid sequence is depicted in SEQ ID NO: 7. Accordingly, as used herein, the term “endostatin” is understood to mean a protein comprising the amino acid sequence of SEQ ID NO: 7, or comprising an amino acid sequence having greater than 80%, 85%, 86%, 87%, 88%, 89%, 90%, 91%, 92%, 93%, 94%, 95%, 96%, 97%, 98%, or 99% sequence identity to SEQ ID NO: 7, or a fragment of any of the forgoing that is capable of noncovalently oligomerizing into trimers, for example, through an association domain present in SEQ ID NO: 7. Oligomerization can be assayed by any method known in the art, including, for example, size exclusion chromatography, analytical ultracentrifugation, scattering techniques, NMR spectroscopy, isothermal titration calorimetry, fluorescence anisotropy and mass spectrometry.

In certain embodiments, a disclosed recombinant virus comprises a nucleotide sequence encoding the amino acid sequence of SEQ ID NO: 7 or SEQ ID NO: 8, or a sequence having 80%, 85%, 86%, 87%, 88%, 89%, 90%, 91%, 92%, 93%, 94%, 95%, 96%, 97%, 98%, or 99% sequence identity to SEQ ID NO: 7 or SEQ ID NO: 8. In certain embodiments, a disclosed recombinant virus comprises the nucleotide sequence of SEQ ID NO: 9 or SEQ ID NO: 10, or a sequence having 80%, 85%, 86%, 87%, 88%, 89%, 90%, 91%, 92%, 93%, 94%, 95%, 96%, 97%, 98%, or 99% sequence identity to SEQ ID NO: 9 or SEQ ID NO: 10.

Angiostatin is a proteolytic fragment of plasminogen. An exemplary human plasminogen amino acid sequence, corresponding to NCBI Reference Sequence NP_000292.1, is depicted in SEQ ID NO: 11.

Angiostatin can result from proteolytic cleavage of plasminogen at different sites. Plasminogen has five kringle domains, which are generally considered responsible for the anti-angiogenic effects of angiostatin. An exemplary amino acid sequence of the first kringle domain of human plasminogen is depicted in SEQ ID NO: 12, an exemplary amino acid sequence of the second kringle domain of human plasminogen is depicted in SEQ ID NO: 13, an exemplary amino acid sequence of the third kringle domain of human plasminogen is depicted in SEQ ID NO: 14, an exemplary amino acid sequence of the fourth kringle domain of human plasminogen is depicted in SEQ ID NO: 15, and an exemplary amino acid sequence of the fifth kringle domain of human plasminogen is depicted in SEQ ID NO: 16. Accordingly, as used herein, the term “angiostatin” is understood to mean a protein comprising the amino acid sequence of SEQ ID NO: 12, SEQ ID NO: 13, SEQ ID NO: 14, SEQ ID NO: 15, or SEQ ID NO: 16, or comprising an amino acid sequence having greater than 80%, 85%, 86%, 87%, 88%, 89%, 90%, 91%, 92%, 93%, 94%, 95%, 96%, 97%, 98%, or 99% sequence identity to SEQ ID NO: 12, SEQ ID NO: 13, SEQ ID NO: 14, SEQ ID NO: 15, or SEQ ID NO: 16, or a fragment of any of the foregoing that is capable of antagonizing endothelial cell migration and/or endothelial cell proliferation. Endothelial cell migration and/or proliferation can be assayed by any method known in the art, including, for example, those described in Guo et al. (2014) METHODS MOL. BIOL. 1135: 393-402.

In certain embodiments, a disclosed recombinant virus comprises a nucleotide sequence encoding the amino acid sequence of SEQ ID NO: 12, SEQ ID NO: 13, SEQ ID NO: 14, SEQ ID NO: 15, SEQ ID NO: 16, or SEQ ID NO: 17, or a sequence having 80%, 85%, 86%, 87%, 88%, 89%, 90%, 91%, 92%, 93%, 94%, 95%, 96%, 97%, 98%, or 99% sequence identity to SEQ ID NO: 12, SEQ ID NO: 13, SEQ ID NO: 14, SEQ ID NO: 15, SEQ ID NO: 16, or SEQ ID NO: 17. In certain embodiments, a disclosed recombinant virus comprises the nucleotide sequence of SEQ ID NO: 18 or SEQ ID NO: 19, or a sequence having 80%, 85%, 86%, 87%, 88%, 89%, 90%, 91%, 92%, 93%, 94%, 95%, 96%, 97%, 98%, or 99% sequence identity to SEQ ID NO: 18 or SEQ ID NO: 19.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: −G, Cost to open gap [Integer]: default=5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; −W, wordsize [Integer]: default=11 for nucleotides/28 for megablast/3 for proteins; −y, Dropoff (X) for blast extensions in bits: default=20 for blastn/7 for others; −X, X dropoff value for gapped alignment (in bits): default=15 for all programs, not applicable to blastn; and −Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

### IV. Methods of Treatment

For therapeutic use, a recombinant virus is preferably combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing recombinant viruses disclosed herein can be presented in a dosage unit form and can be prepared by any suitable method. A pharmaceutical composition should be formulated to be compatible with its intended route of administration. Examples of routes of administration are intravenous (IV), intradermal, inhalation, intraocular, intranasal, transdermal, topical, transmucosal, rectal, oral, parenteral, subcutaneous, intramuscular, ophthalmic, epidural, intratracheal, sublingual, buccal, vaginal, and nasal administration.

An exemplary route of administration is IV infusion. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

The term “effective amount” as used herein refers to the amount of an active component (e.g., the amount of a recombinant virus of the present invention) sufficient to effect beneficial or desired results. An effective amount can be administered in one or more administrations, applications or dosages and is not intended to be limited to a particular formulation or administration route.

In certain embodiments, a therapeutically effective amount of active component is in the range of 0.1 mg/kg to 100 mg/kg, e.g., 1 mg/kg to 100 mg/kg, 1 mg/kg to 10 mg/kg, 1 mg/kg to 5 mg/kg, 10 mg/kg, 7.5 mg/kg, 5 mg/kg, or 2.5 mg/kg. In certain embodiments, a therapeutically effective amount of the recombinant virus is in the range of 10² to 10¹⁵ plaque forming units (pfus), e.g., 10² to 10¹⁰, 10² to 10⁵, 10⁵ to 10¹⁵, 10⁵ to 10¹⁰, or 10¹⁰ to 10¹⁵ plaque forming units. The amount administered will depend on variables such as the type and extent of disease or indication to be treated, the overall health of the subject, the in vivo potency of the active component, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, the half-life of the recombinant virus, and the disease being treated. Exemplary dosing frequencies are once per day, once per week and once every two weeks. A preferred route of administration is parenteral, e.g., intravenous infusion.

The recombinant adenoviruses disclosed herein can be used to treat various medical indications. For example, the recombinant adenoviruses can be used to treat cancers. The cancer cells are exposed to a therapeutically effective amount of the recombinant adenovirus so as to inhibit or reduce proliferation of the cancer cells. The invention provides a method of treating a cancer in a subject. The method comprises administering to the subject an effective amount of a recombinant adenovirus of the invention either alone or in a combination with another therapeutic agent to treat the cancer in the subject. In certain embodiments, administering an effective amount of a recombinant adenovirus to a subject reduces tumor load in that subject by at least 30%, at least 40%, at least 50%, at least 60%, at least 70%, at least 80%, or at least 90%.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma).

In certain embodiments, the cancer is selected from anal cancer, basal cell carcinoma, bladder cancer, bone cancer, brain cancer, breast cancer, carcinoma, cholangiocarcinoma, cervical cancer, colon cancer, colorectal cancer, endometrial cancer, gastroesophageal cancer, gastrointestinal (GI) cancer, gastrointestinal stromal tumor, hepatocellular carcinoma, gynecologic cancer, head and neck cancer, hematologic cancer, kidney cancer, leukemia, liver cancer, lung cancer, lymphoma, melanoma, merkel cell carcinoma, mesothelioma, neuroendocrine cancer, non-small cell lung cancer, ovarian cancer, pancreatic cancer, pediatric cancer, prostate cancer, renal cell carcinoma, sarcoma, skin cancer, small cell lung cancer, squamous cell carcinoma of the skin, stomach cancer, testicular cancer and thyroid cancer.

In certain embodiments, the cancer is selected from gastroesophageal cancer (e.g., gastric or gastro-esophageal junction adenocarcinoma), non-small cell lung cancer (e.g., metastatic NSCLC), colorectal cancer (e.g., metastatic colorectal cancer), ovarian cancer (e.g., platinum-resistant ovarian cancer), leukemia, cervical cancer (e.g., late-stage cervical cancer) brain and central nervous system cancer (e.g., glioblastoma), kidney cancer (e.g., renal cell carcinoma), a sarcoma (e.g., rhabdomyosarcoma, osteosarcoma, and Ewing sarcoma), lymphoma (e.g., Hodgkin and non-Hodgkin), ocular cancer (e.g., choroidal melanoma and retinoblastoma), and von Hippel-Lindau disease.

In certain embodiments, a disclosed method is used to treat a cancer in a pediatric subject. For example, in certain embodiments, the cancer is selected from brain and central nervous system cancer (e.g., astrocytoma, brain stem glioma, craniopharyngioma, desmoplastic infantile ganglioglioma, ependymoma, high-grade glioma, medulloblastoma, atypical teratoid rhabdoid tumor, neuroblastoma), kidney cancer (e.g., Wilms tumor), ocular cancer (e.g., retinoblastoma), a sarcoma (e.g., rhabdomyosarcoma, osteosarcoma, and Ewing sarcoma), liver cancer (e.g., hepatoblastoma and hepatocellular carcinoma), lymphoma (e.g., Hodgkin and non-Hodgkin), leukemia, and a germ cell tumor.

In certain embodiments, a recombinant adenovirus is administered to the subject in combination with one or more therapies, e.g., surgery, radiation, chemotherapy, immunotherapy, hormone therapy, or virotherapy.

In certain embodiments, the recombinant adenovirus is administered in combination with an anti-angiogenic agent. In certain embodiments, the anti-angiogenic agent is selected from aflibercept, an anti-VEGF antibody (e.g., bevacizumab and ranibizumab), sunitinib, pazopanib, sorafenib, regorafenib, vandetanib, cabozantinib, axitinib, tivozanib, linifanib, pegaptanib, spironolactone, indomethacin, thalidomide, interleukin-12, an anti-FGF antibody, a tyrosine kinase inhibitor, an interferon, suramin, a suramin analog, somatostatin, and a somatostatin analog. In certain embodiments, the anti-angiogenic agent is a VEGF inhibitor, e.g., a VEGF inhibitor selected from aflibercept, bevacizumab, ranibizumab, sunitinib, pazopanib, sorafenib, regorafenib, vandetanib, cabozantinib, axitinib, tivozanib and linifanib. In certain embodiments, the recombinant adenovirus is administered in combination with bevacizumab. In certain embodiments, the administration of an anti-angiogenic agent is more effective, e.g., an equivalent effect is seen with a reduced dose of the anti-angiogenic agent than would be seen if the anti-angiogenic agent were administered in the absence of the recombinant adenovirus. For example, in certain embodiments, the recombinant adenovirus is administered in combination with bevacizumab, e.g., bevacizumab administered at a dose of less than 5 mg/kg, less than 4 mg/kg, less than 3 mg/kg, less than 2 mg/kg, less than 1 mg/kg, less than 0.5 mg/kg, from about 0.5 mg/kg to about 5 mg/kg, from about 0.5 mg/kg to about 4 mg/kg, from about 0.5 mg/kg to about 3 mg/kg, from about 0.5 mg/kg to about 2 mg/kg, from about 0.5 mg/kg to about 1 mg/kg, from about 1 mg/kg to about 5 mg/kg, from about 1 mg/kg to about 4 mg/kg, from about 1 mg/kg to about 3 mg/kg, from about 1 mg/kg to about 2 mg/kg, from about 2 mg/kg to about 5 mg/kg, from about 2 mg/kg to about 4 mg/kg, from about 2 mg/kg to about 3 mg/kg, from about 3 mg/kg to about 5 mg/kg, from about 3 mg/kg to about 4 mg/kg, from about 4 mg/kg to about 5 mg/kg, about 5 mg/kg, about 4 mg/kg, about 3 mg/kg, about 2.5 mg/kg, about 2 mg/kg, about 1 mg/kg, or about 0.5 mg/kg.

In certain embodiments, the recombinant adenovirus is administered in combination with a second recombinant adenovirus. In certain embodiments, the second recombinant adenovirus is an oncolytic adenovirus. In certain embodiments, the second recombinant adenovirus comprises a nucleotide sequence encoding a polypeptide, or a fragment thereof, selected from acetylcholine, an androgen-receptor, an anti-PD-1 antibody heavy chain and/or light chain, an anti-PD-L1 antibody heavy chain and/or light chain, BORIS/CTCFL, BRAF, CD19, CD20, CD30, CD80, CD86, CD137, CD137L, CD154, CEA, DKK1/Wnt, EGFRvIII, FGF, gp100, Her-2/neu, ICAM, IL-1, IL-3, IL-4, IL-5, IL-6, IL-8, IL-9, IL-17, IL-23A/p19, p40, IL-24, IL-27, IL-27A/p28, IL-27B/EBI3, IL-35, interferon-gamma, KRAS, MAGE, MAGE-A3, MART1, melan-A, mesothelin, MUC-1, NY-ESO-1, Podocalyxin (Podxl), p53, TGF-β, a TGF-β trap, thymidine kinase, and tyrosinase. In certain embodiments, the second recombinant adenonvirus comprises a nucleotide sequence encoding a cancer antigen derived from 9D7, androgen receptor, a BAGE family protein, β-catenin, BING-4, BRAF, BRCA1/2, a CAGE family protein, calcium-activated chloride channel 2, CD19, CD20, CD30, CDK4, CEA, CML66, CT9, CT10, cyclin-B1, EGFRvIII, Ep-CAM, EphA3, fibronectin, a GAGE family protein, gp100/pme117, Her-2/neu, HPV E6, HPV E7, Ig, immature laminin receptor, a MAGE family protein (e.g., MAGE-A3), MART-1/melan-A, MART2, MC1R, mesothelin, a mucin family protein (e.g., MUC-1), NY-ESO-1/LAGE-1, P.polypeptide, p53, podocalyxin (Podxl), PRAIVIE, a ras family proteins (e.g., KRAS), prostate specific antigen, a SAGE family protein, SAP-1, SSX-2, survivin, TAG-72, TCR, telomerase, TGF-βRII, TRP-1, TRP-2, tyrosinase, or a XAGE family protein.

In certain embodiments, a recombinant adenovirus of the invention is administered in combination with a tyrosine kinase inhibitor, e.g., erlotinib.

In certain embodiments, a recombinant adenovirus of the invention is administered in combination with a checkpoint inhibitor, e.g., an anti-CTLA-4 antibody, an anti-PD-1 antibody, or an anti-PD-L1 antibody. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies include, for example, atezolizumab (Tecentriq®, Genentech), duvalumab (AstraZeneca), MEDI4736, avelumab, and BMS 936559 (Bristol Myers Squibb Co.).

In certain embodiments, a recombinant adenovirus of the invention is administered in combination with an anti-inflammatory agent. In certain embodiments, a recombinant adenovirus of the invention is administered in combination with an anti-inflammatory agent for the treatment of an ocular cancer. Exemplary anti-inflammatory agents include steroidal anti-inflammatory agents (e.g., glucocorticoids (corticosteroids), e.g., hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca), and aldosterone) and non-steroidal anti-inflammatory agents (NSAIDs; e.g., aspirin, choline and magnesium salicylates, choline salicylate, celecoxib, diclofenac potassium, diclofenac sodium, diclofenac sodium with misoprostol, diflunisal, etodolac, fenoprofen calcium, flurbiprofen, ibuprofen, indomethacin, ketoprofen, magnesium salicylate, meclofenamate sodium, mefenamic acid, meloxicam, nabumetone, naproxen, naproxen sodium, oxaprozin, piroxicam, rofecoxib, salsalate, sodium salicylate, sulindac, tolmetin sodium, valdecoxib, and interleukins, e.g., IL-1, IL-4, IL-6, IL-10, IL-11, and IL-13).

The invention provides a method of normalizing vasculature in a subject, i.e., increasing blood flow and/or delivery of oxygen to a tumor in the subject. The method comprises administering to the subject an effective amount of a recombinant adenovirus of the invention either alone or in a combination with another therapeutic agent to normalize vasculature in the subject. In certain embodiments, administering an effective amount of a recombinant adenovirus to a subject increases blood flow and/or delivery of oxygen to a tumor in the subject by at least 30%, at least 40%, at least 50%, at least 60%, at least 70%, at least 80%, or at least 90%. Vascular normalization can be assayed by methods known in the art, including, e.g., contrast enhanced ultrasound (e.g., dynamic contrast enhanced ultrasound) and FLT-PET. Accordingly, the invention also provides a method of increasing the delivery of a therapeutic agent to a tumor. The method comprises administering to the subject an effective amount of a recombinant adenovirus of the invention in a combination with another therapeutic agent to increase the delivery of the therapeutic agent to the tumor. In certain embodiments, administering an effective amount of a recombinant adenovirus in combination with another therapeutic agent increases delivery of the therapeutic agent to the tumor by at least 30%, at least 40%, at least 50%, at least 60%, at least 70%, at least 80%, or at least 90% relative to administrating the therapeutic agent in the absence of the recombinant adenovirus. In certain embodiments, the therapeutic agent is administered concurrently with the recombinant adenovirus or immediately following the recombinant adenovirus.

The invention also provides a method of lowering blood pressure in a subject in need thereof. The method comprises administering to the subject an effective amount of a recombinant adenovirus described herein to lower blood pressure in the subject. As used herein, “blood pressure” may refer to systolic blood pressure, diastolic blood pressure, or the ratio of systolic to diastolic blood pressure. In certain embodiments, administering an effective amount of a recombinant adenovirus to a subject lowers blood pressure by at least 5%, at least 10%, at least 15%, at least 20%, at least 25%, at least 30%, at least 40%, or at least 50% relative to the subject's blood pressure before the recombinant adenovirus is administered. Blood pressure can be assayed by methods known in the art. The invention also provides a method of treating and/or preventing hypertension, i.e., high blood pressure, in a subject. The method comprises administering to the subject an effective amount of a recombinant adenovirus described herein to treat and/or prevent hypertension in the subject.

The invention also provides a method of increasing nitric oxide (NO) production or increasing nitric oxide (NO) levels in a subject in need thereof. The method comprises administering to the subject an effective amount of a recombinant adenovirus described herein to increase NO production or NO levels in the subject. NO plays a major role in regulating blood pressure. NO production or levels may be increased in a cell, body fluid, tissue, organ, or physiological system of the subject. In certain embodiments, NO production or levels are increased in a cell, e.g., an endothelial cell or smooth muscle cell, or a body fluid, e.g., serum. In certain embodiments, administering an effective amount of a recombinant adenovirus to a subject increases NO production or levels in the subject by at least 30%, at least 40%, at least 50%, at least 60%, at least 70%, at least 80%, at least 90%, or at least 100% relative to the NO production or levels before the recombinant adenovirus is administered. NO production can be assayed by methods known in the art, including, e.g., fluorometric methods, e.g., as described in Miles et al. (1996) METHODS ENZYMOL. 268:105-20.

Hypertension is a dose limiting, toxic side effect associated with VEGF inhibitors. Accordingly, in certain embodiments of each of the foregoing methods, the subject is receiving or has received a VEGF inhibitor.

The invention also provides a method of treating an angiogenesis-associated disorder in a subject. The method comprises administering to the subject an effective amount of a recombinant adenovirus of the invention either alone or in a combination with another therapeutic agent to treat the disorder in the subject. As used herein, an angiogenesis associated disorder refers to any disorder associated with overactive or pathogenic angiogenesis. Exemplary angiogenesis-associated disorders include benign tumors, blood-borne tumors, obesity, primary hyperparathyroidism, secondary hyperparathyroidism, tertiary hyperparathyroidism, corneal graft rejection, contact lens overwear, Lyme's disease, Behcet's disease, herpes zoster, syphilis, post-laser complications, sickle cell anemia, atherosclerotic plaque, rheumatoid arthritis, psoriasis, diabetic retinopathy, retinopathy of prematurity, rosacea, keloids, macular degeneration, hemangioma, thyroid hyperplasia, preeclampsia, conjunctival telangiectasia, scleroderma, Crohn's disease, endometriosis, fat cell disease, pyogenic granuloma, flushing, rosacea, angiofibroma, and wound granulation.

The term administered “in combination,” as used herein, is understood to mean that two (or more) different treatments are delivered to the subject during the course of the subject's affliction with the disorder, such that the effects of the treatments on the subject overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, the effective amount of the recombinant adenovirus is identified by measuring an immune response to an antigen in the subject and/or the method of treating the subject further comprises measuring an immune response to an antigen in the subject. Hyperproliferative diseases, e.g., cancers, may be characterized by immunosuppression, and measuring an immune response to an antigen in the subject may be indicative of the level of immunosuppression in the subject. Accordingly, measuring an immune response to an antigen in the subject may be indicative of the efficacy of the treatment and/or the effective amount of the recombinant adenovirus. The immune response to the antigen in the subject may be measured by any method known in the art. In certain embodiments, the immune response to the antigen is measured by injecting the subject with the antigen at an injection site on the skin of the subject and measuring the size of an induration or amount of inflammation at the injection site. In certain embodiments, the immune response to the antigen is measured by release of a cytokine from a cell of the subject (e.g., interferon gamma, IL-4 and/or IL-5) upon exposure to the antigen.

Throughout the description, where viruses, compositions, and systems are described as having, including, or comprising specific components, or where processes and methods are described as having, including, or comprising specific steps, it is contemplated that, additionally, there are compositions, devices, and systems of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a virus, a composition, a system, a method, or a process described herein can be combined in a variety of ways without departing from the spirit and scope of the present invention, whether explicit or implicit herein. For example, where reference is made to a particular virus, that virus can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

At various places in the present specification, viruses, compositions, systems, processes and methods, or features thereof, are disclosed in groups or in ranges. It is specifically intended that the description include each and every individual subcombination of the members of such groups and ranges. By way of other examples, an integer in the range of 1 to 20 is specifically intended to individually disclose 1, 2, 3, 4, 5, 6, 7, 8, 9, 10, 11, 12, 13, 14, 15, 16, 17, 18, 19, and 20.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1: Construction of Endostatin or Angiostatin Expressing Adenoviruses

This Example describes the construction of a recombinant adenovirus type 5 (Ad5) that expresses endostatin and/or angiostatin.

A plasmid carrying the 5′ portion of the adenovirus type 5 genomic sequence was modified to carry the deletion of a nucleotide region located from −304 to −255 upstream of the E1a initiation site, which renders E1a expression cancer-selective (as previously described in U.S. Pat. No. 9,073,980). The modified plasmid is hereafter referred to as the TAV plasmid, and any resulting viral particles produced therefrom are hereafter referred to as the TAV adenovirus.

The TAV plasmid was further modified to carry a SalI site at the start of the E1b-19k region and an XhoI site 200 base pairs 3′ of the SalI site to facilitate insertion of therapeutic transgenes. To delete the 200 base pair E1b-19k region the plasmid was cut with SalI and XhoI and self-ligated. The nucleotide sequence of the modified E1b-19k region is as follows, with the residual bases from the fused SalI and XhoI sites underlined:

### ATCTTGGTTACATCTGACCTCGTCGAGTCACCAGGCGCTTTTCCAA (SEQ ID NO: 24).

The modified plasmid is hereafter referred to as the TAV-Δ19k plasmid, and any resulting viral particles produced therefrom are hereafter referred to as the TAV-Δ19k adenovirus.

A nucleotide sequence encoding amino acid residues 1-26 of mouse collagen XVIII (corresponding to the signal peptide) followed by residues 1577-1774 of mouse collagen XVIII (corresponding to a C-terminal fragment) was cloned in to the modified E1b-19k region of the TAV-Δ19k plasmid. All mouse collagen XVIII amino acid residue numbers are relative to UniProt Reference Sequence: P39061, depicted herein as SEQ ID NO: 25. The modified plasmid is hereafter referred to as the TAV-Endo plasmid, and any resulting viral particles produced therefrom are hereafter referred to as the TAV-Endo adenovirus. The nucleotide sequence of the TAV-Endo plasmid in the E1b-19k region is as follows, where the flanking E1b-19k sequence including the SalI and XhoI restriction sites is underlined:

(SEQ ID NO: 26)
ATCTGACCTCGTCGACATGGCTCCCGACCCCAGCAGACGCCTCTGCCTGC
TGCTGCTGTTGCTGCTCTCCTGCCGCCTTGTGCCTGCCAGCGCTTATGTG
CACCTGCCGCCAGCCCGCCCCACCCTCTCACTTGCTCATACTCATCAGGA
CTTTCAGCCAGTGCTCCACCTGGTGGCACTGAACACCCCCCTGTCTGGAG
GCATGCGTGGTATCCGTGGAGCAGATTTCCAGTGCTTCCAGCAAGCCCGA
GCCGTGGGGCTGTCGGGCACCTTCCGGGCTTTCCTGTCCTCTAGGCTGCA
GGATCTCTATAGCATCGTGCGCCGTGCTGACCGGGGGTCTGTGCCCATCG
TCAACCTGAAGGACGAGGTGCTATCTCCCAGCTGGGACTCCCTGTTTTCT
GGCTCCCAGGGTCAACTGCAACCCGGGGCCCGCATCTTTTCTTTTGACGG
CAGAGATGTCCTGAGACACCCAGCCTGGCCGCAGAAGAGCGTATGGCACG
GCTCGGACCCCAGTGGGCGGAGGCTGATGGAGAGTTACTGTGAGACATGG
CGAACTGAAACTACTGGGGCTACAGGTCAGGCCTCCTCCCTGCTGTCAGG
CAGGCTCCTGGAACAGAAAGCTGCGAGCTGCCACAACAGCTACATCGTCC
TGTGCATTGAGAATAGCTTCATGACCTCTTTCTCCAAATAGCTCGAGTCA
CCAGGCG.

Additionally, a nucleotide sequence encoding amino acid residues 1-19 of mouse plasminogen (corresponding to the signal peptide) followed by residues 96-549 of mouse plasminogen (corresponding to kringle domains 1-5) was cloned in to the modified E1b-19k region of the TAV-Δ19k plasmid. All mouse plasminogen amino acid residue numbers are relative to UniProt Reference Sequence: P20918, depicted herein as SEQ ID NO: 27. The modified plasmid is hereafter referred to as the TAV-Ang plasmid, and any resulting viral particles produced therefrom are hereafter referred to as the TAV-Ang adenovirus. The nucleotide sequence of the TAV-Ang plasmid in the E1b-19k region is as follows, where the flanking E1b-19k sequence including the SalI and XhoI restriction sites is underlined:

(SEQ ID NO: 28)
ATCTGACCTCGTCGACATGGACCACAAGGAAGTAATCCTTCTGTTTCTCT
TGCTTCTGAAACCAGGACAAGGGAAGAGAGTGTATCTGTCAGAATGTAAG
ACCGGCATCGGCAACGGCTACAGAGGAACAATGTCCAGGACAAAGAGTGG
TGTTGCCTGTCAAAAGTGGGGTGCCACGTTCCCCCACGTACCCAACTACT
CTCCCAGTACACATCCCAATGAGGGACTAGAAGAAAATTACTGTAGGAAC
CCAGACAATGATGAACAAGGGCCTTGGTGCTACACTACAGATCCGGACAA
GAGATATGACTACTGCAACATTCCTGAATGTGAAGAAGAATGCATGTACT
GCAGTGGCGAAAAGTATGAGGGGAAAATCTCCAAGACCATGTCTGGACTT
GACTGCCAGGCCTGGGATTCTCAGAGCCCACATGCTCATGGATACATCCC
TGCCAAATTCCCAAGCAAGAACCTGAAGATGAATTATTGCCGCAACCCTG
ACGGGGAGCCAAGGCCCTGGTGCTTCACAACAGACCCCACCAAACGCTGG
GAATACTGTGACATCCCCCGCTGCACAACACCCCCGCCCCCACCCAGCCC
AACCTACCAATGTCTGAAAGGAAGAGGTGAAAATTACCGAGGGACCGTGT
CTGTCACCGTGTCTGGGAAAACCTGTCAGCGCTGGAGTGAGCAAACCCCT
CATAGGCACAACAGGACACCAGAAAATTTCCCCTGCAAAAATCTGGAGGA
GAATTACTGCCGGAACCCGGATGGAGAAACTGCTCCCTGGTGCTATACCA
CTGACAGCCAGCTGAGGTGGGAGTACTGTGAGATTCCATCCTGCGAGTCC
TCAGCATCACCAGACCAGTCAGATTCCTCAGTTCCACCAGAGGAGCAAAC
ACCTGTGGTCCAGGAATGCTACCAGAGCGATGGGCAGAGCTATCGGGGTA
CATCGTCCACTACCATCACAGGGAAGAAGTGCCAGTCCTGGGCAGCTATG
TTTCCACATAGGCATTCGAAGACGCCAGAGAACTTCCCAGATGCTGGCTT
GGAGATGAACTATTGCAGGAACCCGGATGGTGACAAGGGCCCTTGGTGCT
ACACCACTGACCCGAGCGTCAGGTGGGAATACTGCAACCTGAAGCGGTGC
TCAGAGACAGGAGGGAGTGTTGTGGAATTGCCCACAGTTTCCCAGGAACC
AAGTGGGCCGAGCGACTCTGAGACAGACTGCATGTATGGGAATGGCAAAG
ACTACCGGGGCAAAACGGCCGTCACTGCAGCTGGCACCCCTTGCCAAGGA
TGGGCTGCCCAGGAGCCCCACAGGCACAGCATCTTCACCCCACAGACAAA
CCCACGGGCAGGTCTGGAAAAGAATTATTGCCGAAACCCCGATGGGGATG
TGAATGGTCCTTGGTGCTATACAACAAACCCTAGATGATAGCTCGAGTCA
CCAGGCG.

The various plasmids described were used along with other plasmids carrying the remainder of the adenovirus type 5 genomic sequence (based on strain dl309) to generate recombinant adenoviruses.

### Example 2: Construction of Endostatin and/or Angiostatin Expressing Adenoviruses

This Example describes the construction of a recombinant adenovirus type 5 (Ad5) that expresses endostatin and/or angiostatin.

A plasmid carrying the 5′ portion of the adenovirus type 5 genomic sequence is modified to carry the deletion of a nucleotide region located from −304 to −255 upstream of the E1a initiation site, which renders E1a expression cancer-selective (as previously described in U.S. Pat. No. 9,073,980). The modified plasmid is hereafter referred to as the TAV plasmid, and any resulting viral particles produced therefrom are hereafter referred to as the TAV adenovirus.

The TAV plasmid is further modified to carry a SalI site at the start of the E1b-19k region and an XhoI site 200 base pairs 3′ of the SalI site to facilitate insertion of therapeutic transgenes. To delete the 200 base pair E1b-19k region the plasmid is cut with SalI and XhoI and self-ligated. The nucleotide sequence of the modified E1b-19k region is as follows, with the residual bases from the fused SalI and XhoI sites underlined:

(SEQ ID NO: 24)
ATCTTGGTTACATCTGACCTCGTCGAGTCACCAGGCGCTTTTCCAA.

The modified plasmid is hereafter referred to as the TAV-Δ19k plasmid, and any resulting viral particles produced therefrom are hereafter referred to as the TAV-Δ19k adenovirus.

A nucleotide sequence encoding amino acid residues 1-23 of human collagen XVIII (corresponding to the signal peptide) followed by residues 1318-1516 of human collagen XVIII (corresponding to a C-terminal fragment) is cloned in to the modified E1b-19k region of the TAV-Δ19k plasmid. All human collagen XVIII amino acid residue numbers are relative to NCBI Reference Sequence: NP_085059.2, depicted herein as SEQ ID NO: 6. The modified plasmid is hereafter referred to as the TAV-hEndo plasmid, and any resulting viral particles produced therefrom are hereafter referred to as the TAV-hEndo adenovirus. The nucleotide sequence of the TAV-hEndo plasmid in the E1b-19k region is as follows, where the flanking E1b-19k sequence including the SalI and XhoI restriction sites is underlined:

(SEQ ID NO: 9)
ATCTGACCTCGTCGACATGGCTCCCTACCCCTGTGGCTGCCACATCCTGC
TGCTGCTCTTCTGCTGCCTGGCGGCTGCCCGGGCCAGCTCCTACGTGCAC
CTGCGGCCGGCGCGACCCACAAGCCCACCCGCCCACAGCCACCGCGACTT
CCAGCCGGTGCTCCACCTGGTTGCGCTCAACAGCCCCCTGTCAGGCGGCA
TGCGGGGCATCCGCGGGGCCGACTTCCAGTGCTTCCAGCAGGCGCGGGCC
GTGGGGCTGGCGGGCACCTTCCGCGCCTTCCTGTCCTCGCGCCTGCAGGA
CCTGTACAGCATCGTGCGCCGTGCCGACCGCGCAGCCGTGCCCATCGTCA
ACCTCAAGGACGAGCTGCTGTTTCCCAGCTGGGAGGCTCTGTTCTCAGGC
TCTGAGGGTCCGCTGAAGCCCGGGGCACGCATCTTCTCCTTTGACGGCAA
GGACGTCCTGAGGCACCCCACCTGGCCCCAGAAGAGCGTGTGGCATGGCT
CGGACCCCAACGGGCGCAGGCTGACCGAGAGCTACTGTGAGACGTGGCGG
ACGGAGGCTCCCTCGGCCACGGGCCAGGCCTCCTCGCTGCTGGGGGGCAG
GCTCCTGGGGCAGAGTGCCGCGAGCTGCCATCACGCCTACATCGTGCTCT
GCATTGAGAACAGCTTCATGACTGCCTCCAAGTAGCTCGAGTCACCAGGC
G.

Additionally, a nucleotide sequence encoding amino acid residues 1-19 of human plasminogen (corresponding to the signal peptide) followed by residues 97-549 of human plasminogen (corresponding to kringle domains 1-5) is cloned in to the modified E1b-19k region of the TAV-Δ19k plasmid. All human plasminogen amino acid residue numbers are relative to NCBI Reference Sequence: NP_000292.1, depicted herein as SEQ ID NO: 11. The modified plasmid is hereafter referred to as the TAV-hAng plasmid, and any resulting viral particles produced therefrom are hereafter referred to as the TAV-hAng adenovirus. The nucleotide sequence of the TAV-hAng plasmid in the E1b-19k region is as follows, where the flanking E1b-19k sequence including the SalI and XhoI restriction sites is underlined:

(SEQ ID NO: 18)
ATCTGACCTCGTCGACATGGAACATAAGGAAGTGGTTCTTCTACTTCTTTTATTTCTGAAATC
AGGTCAAGGAAAAGTGTATCTCTCAGAGTGCAAGACTGGGAATGGAAAGAACTACAGAGGGAC
GATGTCCAAAACAAAAAATGGCATCACCTGTCAAAAATGGAGTTCCACTTCTCCCCACAGACC
TAGATTCTCACCTGCTACACACCCCTCAGAGGGACTGGAGGAGAACTACTGCAGGAATCCAGA
CAACGATCCGCAGGGGCCCTGGTGCTATACTACTGATCCAGAAAAGAGATATGACTACTGCGA
CATTCTTGAGTGTGAAGAGGAATGTATGCATTGCAGTGGAGAAAACTATGACGGCAAAATTTC
CAAGACCATGTCTGGACTGGAATGCCAGGCCTGGGACTCTCAGAGCCCACACGCTCATGGATA
CATTCCTTCCAAATTTCCAAACAAGAACCTGAAGAAGAATTACTGTCGTAACCCCGATAGGGA
GCTGCGGCCTTGGTGTTTCACCACCGACCCCAACAAGCGCTGGGAACTTTGTGACATCCCCCG
CTGCACAACACCTCCACCATCTTCTGGTCCCACCTACCAGTGTCTGAAGGGAACAGGTGAAAA
CTATCGCGGGAATGTGGCTGTTACCGTGTCCGGGCACACCTGTCAGCACTGGAGTGCACAGAC
CCCTCACACACATAACAGGACACCAGAAAACTTCCCCTGCAAAAATTTGGATGAAAACTACTG
CCGCAATCCTGACGGAAAAAGGGCCCCATGGTGCCATACAACCAACAGCCAAGTGCGGTGGGA
GTACTGTAAGATACCGTCCTGTGACTCCTCCCCAGTATCCACGGAACAATTGGCTCCCACAGC
ACCACCTGAGCTAACCCCTGTGGTCCAGGACTGCTACCATGGTGATGGACAGAGCTACCGAGG
CACATCCTCCACCACCACCACAGGAAAGAAGTGTCAGTCTTGGTCATCTATGACACCACACCG
GCACCAGAAGACCCCAGAAAACTACCCAAATGCTGGCCTGACAATGAACTACTGCAGGAATCC
AGATGCCGATAAAGGCCCCTGGTGTTTTACCACAGACCCCAGCGTCAGGTGGGAGTACTGCAA
CCYGAAAAAATGCTCAGGAACAGAAGCGAGTGTTGTAGCACCTCCGCCTGTTGTCCTGCTTCC
AGATGTAGAGACTCCTTCCGAAGAAGACTGTATGTTTGGGAATGGGAAAGGATACCGAGGCAA
GAGGGCGACCACTGTTACTGGGACGCCATGCCAGGACTGGGCTGCCCAGGAGCCCCATAGACA
CAGCATTTTCACTCCAGAGACAAATCCACGGGCGGGTCTGGAAAAAAATTACTGCCGTAACCC
TGATGGTGATGTAGGTGGTCCCTGGTGCTACACGACAAATCCAAGATAGCTCGAGTCACCAGG
CG.

Additionally, a nucleotide sequence encoding amino acid residues 1-23 of human collagen XVIII (corresponding to the signal peptide) followed by residues 1318-1516 of human collagen XVIII (corresponding to a C-terminal fragment) followed by an encephalomyocarditis virus (EMCV) IRES followed by a nucleotide sequence encoding amino acid residues 1-19 of human plasminogen (corresponding to the signal peptide) followed by residues 97-549 of human plasminogen (corresponding to kringle domains 1-5) is cloned in to the modified E1b-19k region of the TAV-Δ19k plasmid. The modified plasmid is hereafter referred to as the TAV-hEndo-IRES-hAng plasmid, and any resulting viral particles produced therefrom are hereafter referred to as the TAV-hEndo-IRES-hAng adenovirus. The nucleotide sequence of the TAV-hEndo-IRES-hAng plasmid in the E1b-19k region is as follows, where the coding regions are capitalized, the IRES is lowercase, and the flanking E1b-19k sequence including the SalI and XhoI restriction sites is underlined:

(SEQ ID NO: 21)
ATCTGACCTCGTCGACATGGCTCCCTACCCCTGTGGCTGCCACATCCTGCTGCTGCTCTTCTG
CTGCCTGGCGGCTGCCCGGGCCAGCTCCTACGTGCACCTGCGGCCGGCGCGACCCACAAGCCC
ACCCGCCCACAGCCACCGCGACTTCCAGCCGGTGCTCCACCTGGTTGCGCTCAACAGCCCCCT
GTCAGGCGGCATGCGGGGCATCCGCGGGGCCGACTTCCAGTGCTTCCAGCAGGCGCGGGCCGT
GGGGCTGGCGGGCACCTTCCGCGCCTTCCTGTCCTCGCGCCTGCAGGACCTGTACAGCATCGT
GCGCCGTGCCGACCGCGCAGCCGTGCCCATCGTCAACCTCAAGGACGAGCTGCTGTTTCCCAG
CTGGGAGGCTCTGTTCTCAGGCTCTGAGGGTCCGCTGAAGCCCGGGGCACGCATCTTCTCCTT
TGACGGCAAGGACGTCCTGAGGCACCCCACCTGGCCCCAGAAGAGCGTGTGGCATGGCTCGGA
CCCCAACGGGCGCAGGCTGACCGAGAGCTACTGTGAGACGTGGCGGACGGAGGCTCCCTCGGC
CACGGGCCAGGCCTCCTCGCTGCTGGGGGGCAGGCTCCTGGGGCAGAGTGCCGCGAGCTGCCA
TCACGCCTACATCGTGCTCTGCATTGAGAACAGCTTCATGACTGCCTCCAAGTAGtaacgtta
ctggccgaagccgcttggaataaggccggtgtgcgtttgtctatatgttattttccaccatat
tgccgtcttttggcaatgtgagggcccggaaacctggccctgtcttcttgacgagcattccta
ggggtctttcccctctcgccaaaggaatgcaaggtctgttgaatgtcgtgaaggaagcagttc
ctctggaagcttcttgaagacaaacaacgtctgtagcgaccctttgcaggcagcggaaccccc
cacctggcgacaggtgcctctgcggccaaaagccacgtgtataagatacacctgcaaaggcgg
cacaaccccagtgccacgttgtgagttggatagttgtggaaagagtcaaatggctctcctcaa
gcgtattcaacaaggggctgaaggatgcccagaaggtaccccattgtatgggatctgatctgg
ggcctcggtgcacatgctttacatgtgtttagtcgaggttaaaaaacgtctaggccccccgaa
ccacggggacgtggttttcctttgaaaaacacgatgataatATGGAACATAAGGAAGTGGTTC
TTCTACTTCTTTTATTTCTGAAATCAGGTCAAGGAAAAGTGTATCTCTCAGAGTGCAAGACTG
GGAATGGAAAGAACTACAGAGGGACGATGTCCAAAACAAAAAATGGCATCACCTGTCAAAAAT
GGAGTTCCACTTCTCCCCACAGACCTAGATTCTCACCTGCTACACACCCCTCAGAGGGACTGG
AGGAGAACTACTGCAGGAATCCAGACAACGATCCGCAGGGGCCCTGGTGCTATACTACTGATC
CAGAAAAGAGATATGACTACTGCGACATTCTTGAGTGTGAAGAGGAATGTATGCATTGCAGTG
GAGAAAACTATGACGGCAAAATTTCCAAGACCATGTCTGGACTGGAATGCCAGGCCTGGGACT
CTCAGAGCCCACACGCTCATGGATACATTCCTTCCAAATTTCCAAACAAGAACCTGAAGAAGA
ATTACTGTCGTAACCCCGATAGGGAGCTGCGGCCTTGGTGTTTCACCACCGACCCCAACAAGC
GCTGGGAACTTTGTGACATCCCCCGCTGCACAACACCTCCACCATCTTCTGGTCCCACCTACC
AGTGTCTGAAGGGAACAGGTGAAAACTATCGCGGGAATGTGGCTGTTACCGTGTCCGGGCACA
CCTGTCAGCACTGGAGTGCACAGACCCCTCACACACATAACAGGACACCAGAAAACTTCCCCT
GCAAAAATTTGGATGAAAACTACTGCCGCAATCCTGACGGAAAAAGGGCCCCATGGTGCCATA
CAACCAACAGCCAAGTGCGGTGGGAGTACTGTAAGATACCGTCCTGTGACTCCTCCCCAGTAT
CCACGGAACAATTGGCTCCCACAGCACCACCTGAGCTAACCCCTGTGGTCCAGGACTGCTACC
ATGGTGATGGACAGAGCTACCGAGGCACATCCTCCACCACCACCACAGGAAAGAAGTGTCAGT
CTTGGTCATCTATGACACCACACCGGCACCAGAAGACCCCAGAAAACTACCCAAATGCTGGCC
TGACAATGAACTACTGCAGGAATCCAGATGCCGATAAAGGCCCCTGGTGTTTTACCACAGACC
CCAGCGTCAGGTGGGAGTACTGCAACCTGAAAAAATGCTCAGGAACAGAAGCGAGTGTTGTAG
CACCTCCGCCTGTTGTCCTGCTTCCAGATGTAGAGACTCCTTCCGAAGAAGACTGTATGTTTG
GGAATGGGAAAGGATACCGAGGCAAGAGGGCGACCACTGTTACTGGGACGCCATGCCAGGACT
GGGCTGCCCAGGAGCCCCATAGACACAGCATTTTCACTCCAGAGACAAATCCACGGGCGGGTC
TGGAAAAAAATTACTGCCGTAACCCTGATGGTGATGTAGGTGGTCCCTGGTGCTACACGACAA
ATCCAAGATAGCTCGAGTCACCAGGCG.

Additionally, a nucleotide sequence encoding amino acid residues 1-26 of mouse collagen XVIII (corresponding to the signal peptide) followed by residues 1577-1774 of mouse collagen XVIII (corresponding to a C-terminal fragment) followed by an encephalomyocarditis virus (EMCV) IRES followed by a nucleotide sequence encoding amino acid residues 1-19 of mouse plasminogen (corresponding to the signal peptide) followed by residues 96-549 of mouse plasminogen (corresponding to kringle domains 1-5) is cloned in to the modified E1b-19k region of the TAV-Δ19k plasmid. The modified plasmid is hereafter referred to as the TAV-Endo-IRES-Ang plasmid, and any resulting viral particles produced therefrom are hereafter referred to as the TAV-Endo-IRES-Ang adenovirus. The nucleotide sequence of the TAV-Endo-IRES-Ang plasmid in the E1b-19k region is as follows, where the coding regions are capitalized, the IRES is lowercase, and the flanking E1b-19k sequence including the SalI and XhoI restriction sites is underlined:

(SEQ ID NO: 29)
ATCTGACCTCGTCGACATGGCTCCCGACCCCAGCAGACGCCTCTGCCTGCTGCTGCTGTTGCT
GCTCTCCTGCCGCCTTGTGCCTGCCAGCGCTTATGTGCACCTGCCGCCAGCCCGCCCCACCCT
CTCACTTGCTCATACTCATCAGGACTTTCAGCCAGTGCTCCACCTGGTGGCACTGAACACCCC
CCTGTCTGGAGGCATGCGTGGTATCCGTGGAGCAGATTTCCAGTGCTTCCAGCAAGCCCGAGC
CGTGGGGCTGTCGGGCACCTTCCGGGCTTTCCTGTCCTCTAGGCTGCAGGATCTCTATAGCAT
CGTGCGCCGTGCTGACCGGGGGTCTGTGCCCATCGTCAACCTGAAGGACGAGGTGCTATCTCC
CAGCTGGGACTCCCTGTTTTCTGGCTCCCAGGGTCAACTGCAACCCGGGGCCCGCATCTTTTC
TTTTGACGGCAGAGATGTCCTGAGACACCCAGCCTGGCCGCAGAAGAGCGTATGGCACGGCTC
GGACCCCAGTGGGCGGAGGCTGATGGAGAGTTACTGTGAGACATGGCGAACTGAAACTACTGG
GGCTACAGGTCAGGCCTCCTCCCTGCTGTCAGGCAGGCTCCTGGAACAGAAAGCTGCGAGCTG
CCACAACAGCTACATCGTCCTGTGCATTGAGAATAGCTTCATGACCTCTTTCTCCAAATAGta
acgttactggccgaagccgcttggaataaggccggtgtgcgtttgtctatatgttattttcca
ccatattgccgtcttttggcaatgtgagggcccggaaacctggccctgtcttcttgacgagca
ttcctaggggtctttcccctctcgccaaaggaatgcaaggtctgttgaatgtcgtgaaggaag
cagttcctctggaagcttcttgaagacaaacaacgtctgtagcgaccctttgcaggcagcgga
accccccacctggcgacaggtgcctctgcggccaaaagccacgtgtataagatacacctgcaa
aggcggcacaaccccagtgccacgttgtgagttggatagttgtggaaagagtcaaatggctct
cctcaagcgtattcaacaaggggctgaaggatgcccagaaggtaccccattgtatgggatctg
atctggggcctcggtgcacatgctttacatgtgtttagtcgaggttaaaaaacgtctaggccc
cccgaaccacggggacgtggttttcctttgaaaaacacgatgataatATGGACCACAAGGAAG
TAATCCTTCTGTTTCTCTTGCTTCTGAAACCAGGACAAGGGAAGAGAGTGTATCTGTCAGAAT
GTAAGACCGGCATCGGCAACGGCTACAGAGGAACAATGTCCAGGACAAAGAGTGGTGTTGCCT
GTCAAAAGTGGGGTGCCACGTTCCCCCACGTACCCAACTACTCTCCCAGTACACATCCCAATG
AGGGACTAGAAGAAAATTACTGTAGGAACCCAGACAATGATGAACAAGGGCCTTGGTGCTACA
CTACAGATCCGGACAAGAGATATGACTACTGCAACATTCCTGAATGTGAAGAAGAATGCATGT
ACTGCAGTGGCGAAAAGTATGAGGGGAAAATCTCCAAGACCATGTCTGGACTTGACTGCCAGG
CCTGGGATTCTCAGAGCCCACATGCTCATGGATACATCCCTGCCAAATTCCCAAGCAAGAACC
TGAAGATGAATTATTGCCGCAACCCTGACGGGGAGCCAAGGCCCTGGTGCTTCACAACAGACC
CCACCAAACGCTGGGAATACTGTGACATCCCCCGCTGCACAACACCCCCGCCCCCACCCAGCC
CAACCTACCAATGTCTGAAAGGAAGAGGTGAAAATTACCGAGGGACCGTGTCTGTCACCGTGT
CTGGGAAAACCTGTCAGCGCTGGAGTGAGCAAACCCCTCATAGGCACAACAGGACACCAGAAA
ATTTCCCCTGCAAAAATCTGGAGGAGAATTACTGCCGGAACCCGGATGGAGAAACTGCTCCCT
GGTGCTATACCACTGACAGCCAGCTGAGGTGGGAGTACTGTGAGATTCCATCCTGCGAGTCCT
CAGCATCACCAGACCAGTCAGATTCCTCAGTTCCACCAGAGGAGCAAACACCTGTGGTCCAGG
AATGCTACCAGAGCGATGGGCAGAGCTATCGGGGTACATCGTCCACTACCATCACAGGGAAGA
AGTGCCAGTCCTGGGCAGCTATGTTTCCACATAGGCATTCGAAGACGCCAGAGAACTTCCCAG
ATGCTGGCTTGGAGATGAACTATTGCAGGAACCCGGATGGTGACAAGGGCCCTTGGTGCTACA
CCACTGACCCGAGCGTCAGGTGGGAATACTGCAACCTGAAGCGGTGCTCAGAGACAGGAGGGA
GTGTTGTGGAATTGCCCACAGTTTCCCAGGAACCAAGTGGGCCGAGCGACTCTGAGACAGACT
GCATGTATGGGAATGGCAAAGACTACCGGGGCAAAACGGCCGTCACTGCAGCTGGCACCCCTT
GCCAAGGATGGGCTGCCCAGGAGCCCCACAGGCACAGCATCTTCACCCCACAGACAAACCCAC
GGGCAGGTCTGGAAAAGAATTATTGCCGAAACCCCGATGGGGATGTGAATGGTCCTTGGTGCT
ATACAACAAACCCTAGATGATAGCTCGAGTCACCAGGCG.

The various plasmids described are used along with other plasmids carrying the remainder of the adenovirus type 5 genomic sequence (based on strain dl309) to generate recombinant adenoviruses.

### Example 3: Anti-Cancer Activity of Endostatin or Angiostatin Expressing Adenoviruses

This example describes the anti-cancer activity of endostatin or angiostatin expressing recombinant adenoviruses produced as described in Example 1.

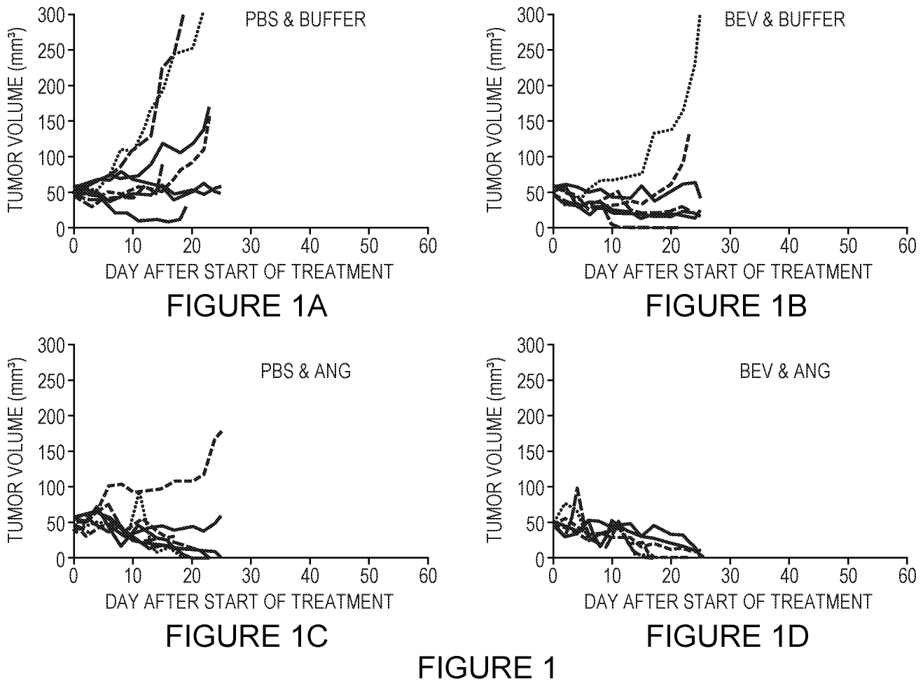

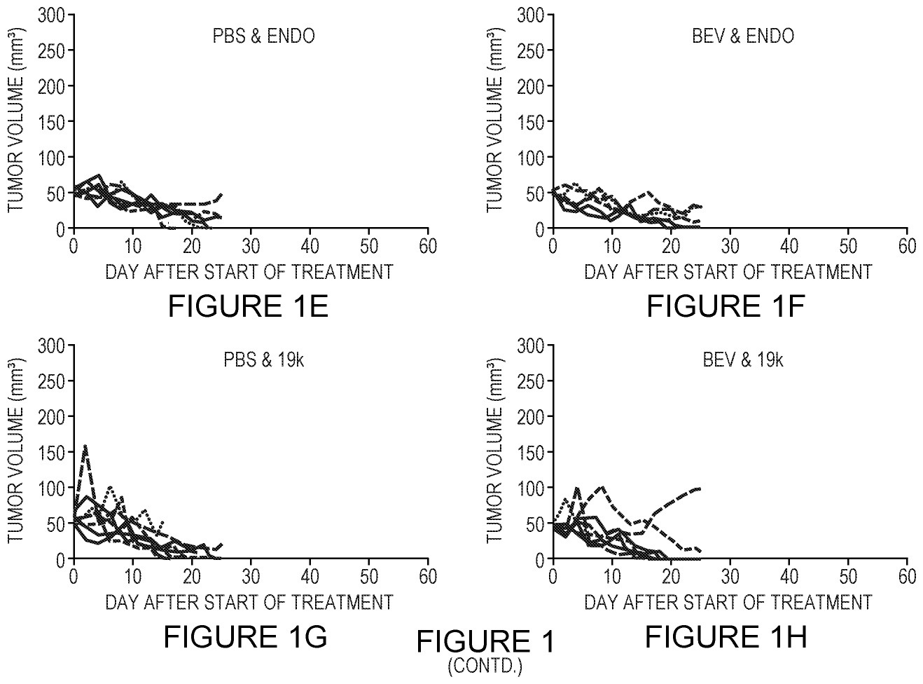

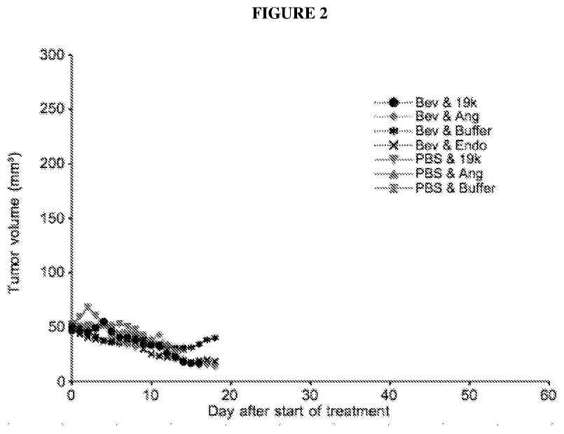

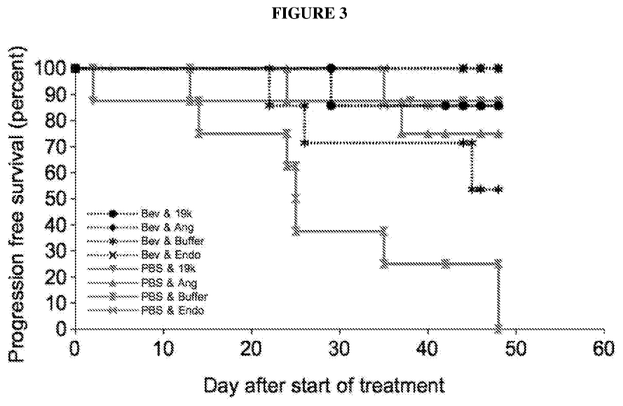

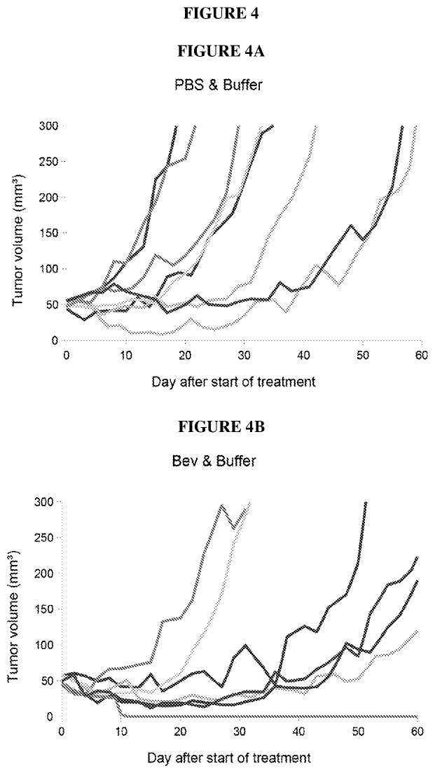

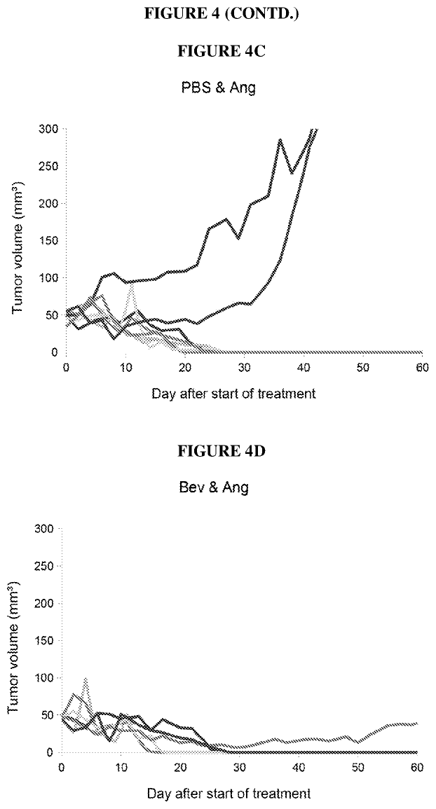

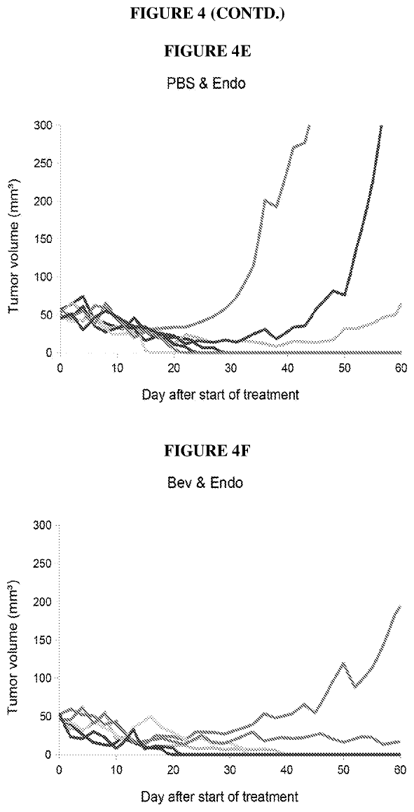

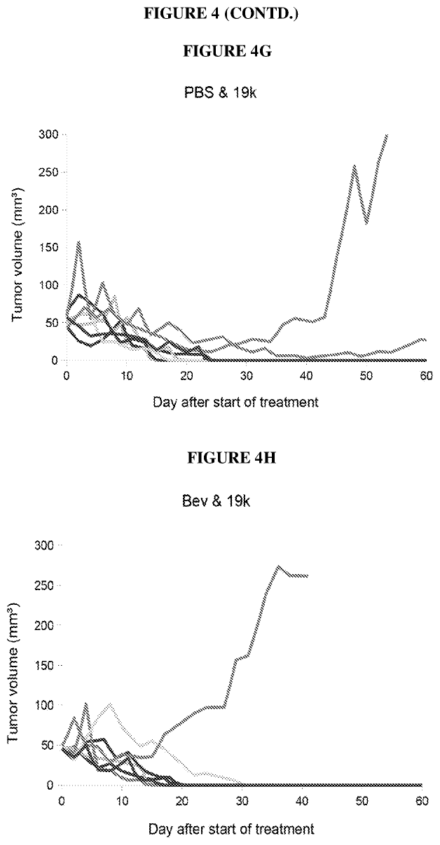

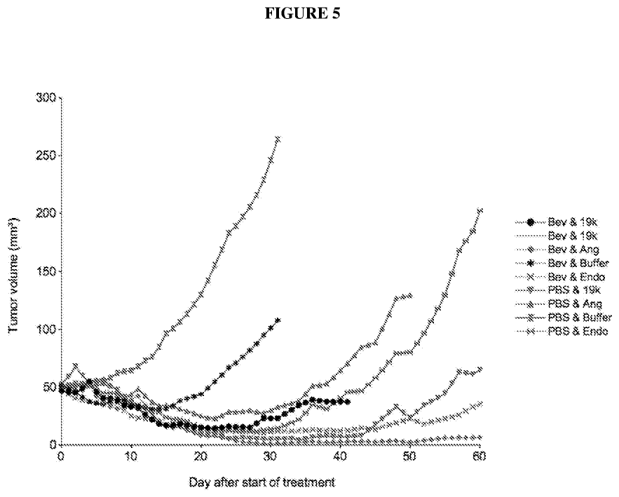

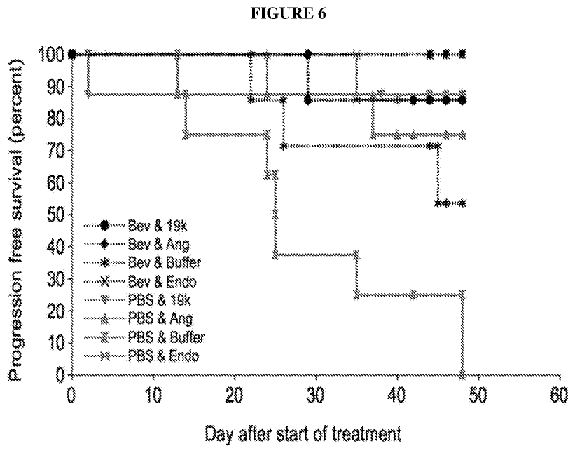

129S4 mice carrying ADS-12 tumors were treated with three intratumoral injections of buffer, TAV-Δ19k, TAV-Endo, or TAV-Ang adenoviruses at 1×10⁹ PFU/dose on days 0, 4, and 8, and/or four intraperitoneal injections of phosphate buffered saline (PBS) or a mouse orthologue of bevacizumab (Bev) on days 1, 5, 9, and 13. Initial results, including tumor volume and progression free survival, are depicted in FIGS. 1-3. Further results after tracking the mice for a longer duration of time are depicted in FIGS. 4-6.

These results demonstrate that the endostatin and angiostatin expressing adenoviruses were effective in reducing tumor volume, and that the endostatin and angiostatin expressing adenoviruses and bevacizumab act synergistically to reduce tumor burden. Surprisingly for an anti-angiogenic treatment, certain mice showed complete remission in tumor volume, rather than merely a delay in tumor growth. These results are particularly surprising because the effects of bevacizumab are cytostatic rather than cytotoxic. Additionally, mice had no evidence of tissue toxicity, as observed by overall appearance, level of activity, and signs of distress (e.g., hunched posture or ruffled fur).

### Example 4: Anti-Cancer Activity of Angiostatin Expressing Adenoviruses

This example describes the anti-cancer activity of angiostatin expressing recombinant adenoviruses produced as described in Example 1.

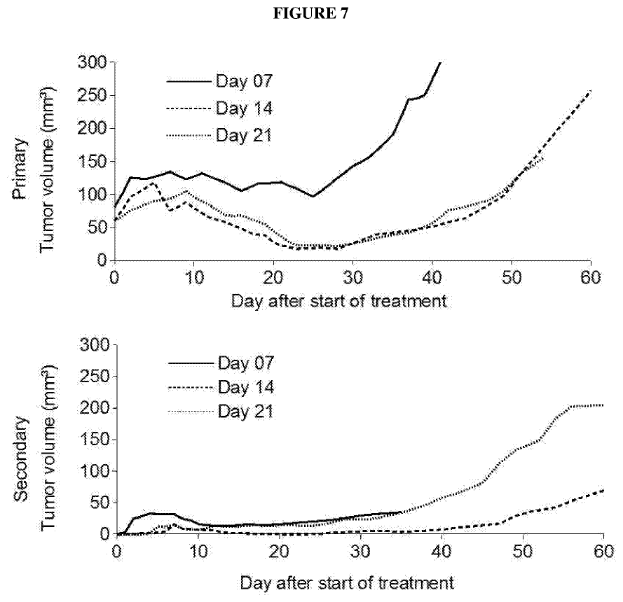

129S4 mice were injected with 1×10⁶ ADS-12 tumor cells on one side of the flank, and primary tumors were allowed to grow to 260-500 mm³. Upon primary tumors reaching target volume (day 0), mice were treated with intratumoral injections of TAV-Ang adenoviruses on days 0, 4, and 8 at 1×10⁹ PFU/dose, following which primary tumor volume was monitored. Upon primary tumors reaching target volume (day 0), mice were additionally injected with 1×10⁶ ADS-12 tumor cells on the opposite side of the flank on days 7, 14, or 21 and the formation, and volume, of secondary tumors on this side of the flank was monitored. Secondary tumors did not receive direct treatment. Results are depicted in FIG. 7, and show that despite no direct treatment, secondary tumors mostly regressed or did not develop at all.

These results show that the angiostatin expressing adenoviruses described herein are effective in reducing contralateral tumor volume.

### Example 5: Anti-Cancer Activity of Adenoviruses

This example describes the anti-cancer activity of recombinant adenoviruses produced as described in Example 1.

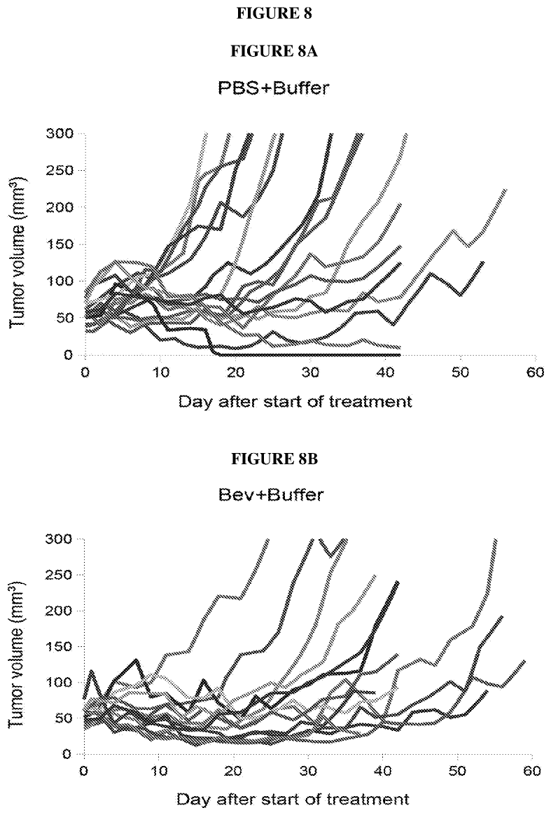

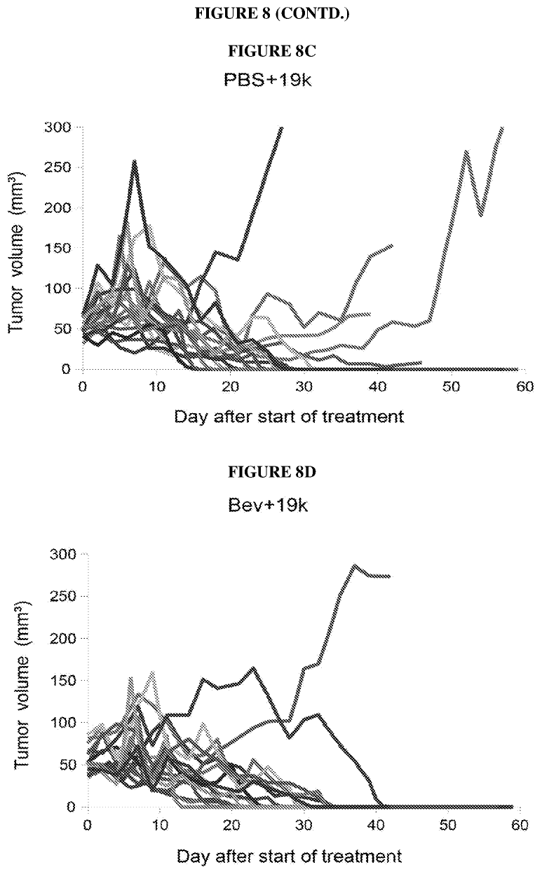

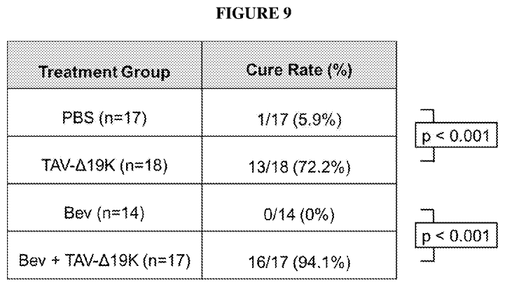

129S4 mice carrying ADS-12 tumors were treated with three intratumoral injections of buffer or TAV-Δ19k at 1×10⁹ PFU/dose on days 0, 4, and 8, and/or four intraperitoneal injections of phosphate buffered saline (PBS) or a mouse orthologue of bevacizumab (Bev) on days 1, 5, 9, and 13. Tumor volumes for each treatment are shown in FIG. 8. Complete tumor regression (cure rates) are shown in FIG. 9. Surprisingly for an anti-angiogenic treatment, certain mice showed complete remission in tumor volume, rather than merely a delay in tumor growth. These results are particularly surprising because the effects of bevacizumab are cytostatic rather than cytotoxic.

These results show that oncolytic adenoviruses, including TAV-Δ19k, alone and in combination with bevacizumab are effective in reducing tumor volume and that oncolytic adenoviruses, including TAV-Δ19k, alone and in combination with bevacizumab can result in complete tumor regression.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and the range of equivalency of the claims are intended to be embraced therein.

What is claimed is:

1. A recombinant adenovirus comprising a first nucleotide sequence encoding a first therapeutic transgene selected from endostatin and angiostatin inserted into an E1b-19K insertion site, wherein the E1b-19K insertion site is located between the start site of E1b-19K and the start site of E1b-55K.

2. The recombinant adenovirus of claim 1, wherein the recombinant adenovirus is a type 5 adenovirus (Ad5).

3. The recombinant adenovirus of claim 1 or 2, wherein the E1b-19K insertion site is located between the start site of E1b-19K and the stop site of E1b-19K.

4. The recombinant adenovirus of any one of claims 1-3, wherein the E1b-19K insertion site comprises a deletion of from about 100 to about 305, about 100 to about 300, about 100 to about 250, about 100 to about 200, about 100 to about 150, about 150 to about 305, about 150 to about 300, about 150 to about 250, or about 150 to about 200 nucleotides adjacent the start site of E1b-19K.

5. The recombinant adenovirus of any one of claims 1-4, wherein the E1b-19K insertion site comprises a deletion of about 200 nucleotides adjacent the start site of E1b-19K.

6. The recombinant adenovirus of any one of claims 1-5, wherein the E1b-19K insertion site comprises a deletion of 202 nucleotides adjacent the start site of E1b-19K.

7. The recombinant adenovirus of any one of claims 1-5, wherein the E1b-19K insertion site comprises a deletion of 203 nucleotides adjacent the start site of E1b-19K.

8. The recombinant adenovirus of any one of claims 1-7, wherein the E1b-19K insertion site comprises a deletion corresponding to nucleotides 1714-1916 of the Ad5 genome (SEQ ID NO: 1).

9. The recombinant adenovirus of any one of claims 1-8, wherein the first therapeutic transgene is inserted between nucleotides corresponding to 1713 and 1917 of the Ad5 genome (SEQ ID NO: 1).

10. The recombinant adenovirus of any one of claims 1-9, wherein the first therapeutic transgene is inserted between CTGACCTC (SEQ ID NO: 2) and TCACCAGG (SEQ ID NO: 3).

11. The recombinant adenovirus of any one of claims 1-10, wherein the recombinant adenovirus comprises, in a 5′ to 3′ orientation, CTGACCTC (SEQ ID NO: 2), the first therapeutic transgene, and TCACCAGG (SEQ ID NO: 3).

12. The recombinant adenovirus of any one of claims 1-11, wherein the recombinant adenovirus comprises a second nucleotide sequence encoding a second therapeutic transgene selected from endostatin and angiostatin.

13. The recombinant adenovirus of claim 12, wherein the second therapeutic transgene is inserted into the E1b-19k insertion site, and the first nucleotide sequence and the second nucleotide sequence are separated by an internal ribosome entry site (IRES).

14. The recombinant adenovirus of claim 13, wherein the IRES is selected from an encephalomyocarditis virus IRES, a foot-and-mouth disease virus IRES, and a poliovirus IRES.

15. The recombinant adenovirus of claim 14, wherein the IRES is an encephalomyocarditis virus IRES.

16. The recombinant adenovirus of claim 15, wherein the IRES comprises SEQ ID NO: 20.

17. The recombinant adenovirus of any one of claims 13-16, wherein the first and second therapeutic transgenes are inserted between nucleotides corresponding to 1713 and 1917 of the Ad5 genome (SEQ ID NO: 1).

18. The recombinant adenovirus of any one of claims 13-17, wherein the first and second therapeutic transgenes are inserted between CTGACCTC (SEQ ID NO: 2) and TCACCAGG (SEQ ID NO: 3).

19. The recombinant adenovirus of any one of claims 13-18, wherein the recombinant adenovirus comprises, in a 5′ to 3′ orientation, CTGACCTC (SEQ ID NO: 2), the first therapeutic transgene, the IRES, the second therapeutic transgene, and TCACCAGG (SEQ ID NO: 3).

20. The recombinant adenovirus of any of claims 1-19, wherein the recombinant adenovirus further comprises an E3 deletion, wherein the E3 deletion is located between the stop site of pVIII and the start site of Fiber.

21. The recombinant adenovirus of claim 20, wherein the E3 deletion is located between the stop site of E3-10.5K and the stop site of E3-14.7K.

22. The recombinant adenovirus of claim 20 or 21, wherein the E3 deletion comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or from about 3000 to about 3185 nucleotides.

23. The recombinant adenovirus of any one of claims 20-22, wherein the E3 deletion comprises a deletion of from about 500 to about 1551, from about 500 to about 1500, from about 500 to about 1000, from about 1000 to about 1551, from about 1000 to about 1500, or from about 1500 to about 1551 nucleotides adjacent the stop site of E3-10.5K.

24. The recombinant adenovirus of any one of claims 20-23, wherein the E3 deletion comprises a deletion of about 1050 nucleotides adjacent the stop site of E3-10.5K.

25. The recombinant adenovirus of any one of claims 20-24, wherein the E3 deletion comprises a deletion of 1063 nucleotides adjacent the stop site of E3-10.5K.

26. The recombinant adenovirus of any one of claims 20-24, wherein the E3 deletion comprises a deletion of 1064 nucleotides adjacent the stop site of E3-10.5K.

27. The recombinant adenovirus of any one of claims 20-26, wherein the E3 deletion comprises a deletion corresponding to the Ad5 dl309 E3 deletion.

28. The recombinant adenovirus of any one of claims 20-27, wherein the E3 deletion comprises a deletion corresponding to nucleotides 29773-30836 of the Ad5 genome (SEQ ID NO: 1).

29. The recombinant adenovirus of claim 12, wherein the second therapeutic transgene is inserted into an E3 insertion site, wherein the E3 insertion site is located between the stop site of pVIII and the start site of Fiber.

30. The recombinant adenovirus of claim 29, wherein the E3 insertion site is located between the stop site of E3-10.5K and the stop site of E3-14.7K.

31. The recombinant adenovirus of claim 29 or 30, wherein the E3 insertion site comprises a deletion of from about 500 to about 3185, from about 500 to about 3000, from about 500 to about 2500, from about 500 to about 2000, from about 500 to about 1500, from about 500 to about 1000, from about 1000 to about 3185, from about 1000 to about 3000, from about 1000 to about 2500, from about 1000 to about 2000, from about 1000 to about 1500, from about 1500 to about 3185, from about 1500 to about 3000, from about 1500 to about 2000, from about 2000 to about 3185, from about 2000 to about 3000, from about 2000 to about 2500, from about 2500 to about 3185, from about 2500 to about 3000, or from about 3000 to about 3185 nucleotides.

32. The recombinant adenovirus of any one of claims 29-31, wherein the E3 insertion site comprises a deletion of from about 500 to about 1551, from about 500 to about 1500, from about 500 to about 1000, from about 1000 to about 1551, from about 1000 to about 1500, or from about 1500 to about 1551 nucleotides adjacent the stop site of E3-10.5K.

33. The recombinant adenovirus of any one of claims 29-32, wherein the E3 insertion site comprises a deletion of about 1050 nucleotides adjacent the stop site of E3-10.5K.

34. The recombinant adenovirus of any one of claims 29-33, wherein the E3 insertion site comprises a deletion of 1063 nucleotides adjacent the stop site of E3-10.5K.

35. The recombinant adenovirus of any one of claims 29-34, wherein the E3 insertion site comprises a deletion of 1064 nucleotides adjacent the stop site of E3-10.5K.

36. The recombinant adenovirus of any one of claims 29-35, wherein the E3 insertion site comprises a deletion corresponding to the Ad5 dl309 E3 deletion.

37. The recombinant adenovirus of any one of claims 29-36, wherein the E3 insertion site comprises a deletion corresponding to nucleotides 29773-30836 of the Ad5 genome (SEQ ID NO: 1).

38. The recombinant adenovirus of any one of claims 29-37, wherein the second therapeutic transgene is inserted between nucleotides corresponding to 29773 and 30836 of the Ad5 genome (SEQ ID NO: 1).

39. The recombinant adenovirus of any one of claims 29-38, wherein the second therapeutic transgene is inserted between CAGTATGA (SEQ ID NO: 4) and TAATAAAAAA (SEQ ID NO: 5).

40. The recombinant adenovirus of any one of claims 29-39, wherein the recombinant adenovirus comprises, in a 5′ to 3′ orientation, CAGTATGA (SEQ ID NO: 4), the second therapeutic transgene, and TAATAAAAAA (SEQ ID NO: 5).

41. The recombinant adenovirus of any one of claims 1-40, wherein the recombinant adenovirus comprises a nucleotide sequence encoding the amino acid sequence of SEQ ID NO: 7, or a nucleotide sequence encoding an amino acid sequence having 80%, 85%, 86%, 87%, 88%, 89%, 90%, 91%, 92%, 93%, 94%, 95%, 96%, 97%, 98%, or 99% sequence identity to SEQ ID NO: 7.

42. The recombinant adenovirus of any one of claims 1-41, wherein the recombinant adenovirus comprises a nucleotide sequence encoding the amino acid sequence of SEQ ID NO: 8, or a nucleotide sequence encoding an amino acid sequence having 80%, 85%, 86%, 87%, 88%, 89%, 90%, 91%, 92%, 93%, 94%, 95%, 96%, 97%, 98%, or 99% sequence identity to SEQ ID NO: 8.

43. The recombinant adenovirus of any one of claims 1-42, wherein the recombinant adenovirus comprises the nucleotide sequence of SEQ ID NO: 9, or comprises a sequence having 80%, 85%, 86%, 87%, 88%, 89%, 90%, 91%, 92%, 93%, 94%, 95%, 96%, 97%, 98%, or 99% sequence identity to SEQ ID NO: 9.

44. The recombinant adenovirus of any one of claims 1-43, wherein the recombinant adenovirus comprises a nucleotide sequence encoding an amino acid sequence selected from SEQ ID NO: 12, SEQ ID NO: 13, SEQ ID NO: 14, SEQ ID NO: 15, and SEQ ID NO: 16, or a nucleotide sequence encoding an amino acid sequence having 80%, 85%, 86%, 87%, 88%, 89%, 90%, 91%, 92%, 93%, 94%, 95%, 96%, 97%, 98%, or 99% sequence identity to an amino acid sequence selected from SEQ ID NO: 12, SEQ ID NO: 13, SEQ ID NO: 14, SEQ ID NO: 15, and SEQ ID NO: 16.

45. The recombinant adenovirus of any one of claims 1-44, wherein the recombinant adenovirus comprises a nucleotide sequence encoding the amino acid sequence of SEQ ID NO: 17, or a nucleotide sequence encoding an amino acid sequence having 80%, 85%, 86%, 87%, 88%, 89%, 90%, 91%, 92%, 93%, 94%, 95%, 96%, 97%, 98%, or 99% sequence identity to SEQ ID NO: 17.

46. The recombinant adenovirus of any one of claims 1-45, wherein the recombinant adenovirus comprises the nucleotide sequence of SEQ ID NO: 18, or comprises a sequence having 80%, 85%, 86%, 87%, 88%, 89%, 90%, 91%, 92%, 93%, 94%, 95%, 96%, 97%, 98%, or 99% sequence identity to SEQ ID NO: 18.

47. The recombinant adenovirus of any one of claims 1-46, wherein the first and/or second therapeutic transgenes are not operably linked to an exogenous promoter sequence.

48. The recombinant adenovirus of claim 47, wherein neither of the therapeutic transgenes are operably linked to an exogenous promoter sequence.

49. The recombinant adenovirus of any one of claims 1-48, wherein the recombinant adenovirus further comprises a deletion of a Pea3 binding site, or a functional fragment thereof.

50. The recombinant adenovirus of claim 49, wherein the recombinant adenovirus comprises a deletion of nucleotides corresponding to about −300 to about −250 upstream of the initiation site of E1a.

51. The recombinant adenovirus of claim 49 or 50, wherein the recombinant adenovirus comprises a deletion of nucleotides corresponding to −304 to −255 upstream of the initiation site of E1a.

52. The recombinant adenovirus of claim 49 or 50, wherein the recombinant adenovirus comprises a deletion of nucleotides corresponding to −305 to −255 upstream of the initiation site of E1a.

53. The recombinant adenovirus of any one of claims 49-52, wherein the recombinant adenovirus comprises a deletion of nucleotides corresponding to 195-244 of the Ad5 genome (SEQ ID NO: 1).

54. The recombinant adenovirus of any one of claims 49-53, wherein the recombinant adenovirus comprises the sequence GGTGTTTTGG (SEQ ID NO: 22).

55. The recombinant adenovirus of any one of claims 49-54, wherein the recombinant adenovirus does not comprise a deletion of an E2F binding site.

56. The recombinant adenovirus of any one of claims 1-48, wherein the recombinant adenovirus further comprises a deletion of a E2F binding site, or a functional fragment thereof.

57. The recombinant adenovirus of claim 56, wherein the recombinant adenovirus does not comprise a deletion of a Pea3 binding site, or a functional fragment thereof.

58. The recombinant adenovirus of any one of claims 1-57, wherein the recombinant adenovirus comprises an E1a promoter having a deletion of a functional TATA box.

59. The recombinant adenovirus of claim 58, wherein the deletion comprises a deletion of the entire TATA box.

60. The recombinant adenovirus of claim 58 or 59, wherein the deletion comprises a deletion of nucleotides corresponding to −27 to −24 of the E1a promoter.

61. The recombinant adenovirus of any one of claims 58-60, wherein the deletion comprises a deletion of nucleotides corresponding to −31 to −24 of the E1a promoter.

62. The recombinant adenovirus of any one of claims 58-61, wherein the deletion comprises a deletion of nucleotides corresponding to −44 to +54 of the E1a promoter.

63. The recombinant adenovirus of any one of claims 58-62, wherein the deletion comprises a deletion of nucleotides corresponding to −146 to +54 of the E1a promoter.

64. The recombinant adenovirus of any one of claims 58-63, wherein the deletion comprises a deletion of nucleotides corresponding to 472 to 475 of the Ad5 genome (SEQ ID NO: 1).

65. The recombinant adenovirus of any one of claims 58-64, wherein the deletion comprises a deletion of nucleotides corresponding to 468 to 475 of the Ad5 genome (SEQ ID NO: 1).

66. The recombinant adenovirus of any one of claims 58-65, wherein the deletion comprises a deletion of nucleotides corresponding to 455 to 552 of the Ad5 genome (SEQ ID NO: 1).

67. The recombinant adenovirus of any one of claims 58-66, wherein the deletion comprises a deletion of nucleotides corresponding to 353-552 of the Ad5 genome (SEQ ID NO: 1).

68. The recombinant adenovirus of any one of claims 58-67, wherein the adenovirus comprises a polynucleotide deletion that results in an adenovirus comprising the sequence CTAGGACTG (SEQ ID NO: 23), AGTGCCCG (SEQ ID NO: 30) and/or TATTCCCG (SEQ ID NO: 31).

69. The recombinant adenovirus of any one of claims 58-68, wherein the E1a promoter comprises the sequence CTAGGACTG (SEQ ID NO: 23).

70. The recombinant adenovirus of any one of claims 1-69, wherein the recombinant adenovirus comprises an E1a promoter having a deletion of a functional CAAT box.

71. The recombinant adenovirus of claim 70, wherein the deletion comprises a deletion of the entire CAAT box.

72. The recombinant adenovirus of claim 70 or 71, wherein the deletion comprises a deletion of nucleotides corresponding to −76 to −68 of the E1a promoter.

73. The recombinant adenovirus of any one of claims 70-72, wherein the deletion comprises a deletion of nucleotides corresponding to 423 to 431 of the Ad5 genome (SEQ ID NO: 1).

74. The recombinant adenovirus of any one of claims 70-73, wherein the adenovirus comprises a polynucleotide deletion that results in an adenovirus comprising the sequence TTCCGTGGCG (SEQ ID NO: 32).

75. The recombinant adenovirus of any one of claims 1-74, wherein the recombinant adenovirus selectively replicates in a hyperproliferative cell.

76. The recombinant adenovirus of any one of claims 1-75, wherein the recombinant adenovirus selectively expresses endostatin and/or angiostatin in a hyperproliferative cell.

77. The recombinant adenovirus of claim 75 or 76, wherein the hyperproliferative cell is a cancer cell.

78. The recombinant adenovirus of any one of claims 1-77, wherein the recombinant adenovirus is an oncolytic adenovirus.

79. A pharmaceutical composition comprising the recombinant adenovirus of any one of claims 1-78 and at least one pharmaceutically acceptable carrier or diluent.

80. A method of expressing endostatin and/or angiostatin in a target cell comprising exposing the cell to an effective amount of the recombinant adenovirus of any one of claims 1-78 to express endostatin and/or angiostatin.

81. A method of inhibiting proliferation of a tumor cell comprising exposing the cell to an effective amount of the recombinant adenovirus of any one of claims 1-78 to inhibit proliferation of the tumor cell.

82. A method of inhibiting tumor growth in a subject in need thereof, the method comprising administering to the subject to an effective amount of the recombinant adenovirus of any one of claims 1-78 to inhibit growth of the tumor.

83. A method of treating cancer in a subject in need thereof, the method comprising administering to the subject an effective amount of the recombinant adenovirus of any one of claims 1-78 to treat the cancer in the subject.

84. The method of claim 83, wherein the recombinant adenovirus is administered in combination with an anti-angiogenic agent.

85. The method of claim 83 or 84, wherein the recombinant adenovirus is administered in combination with one or more therapies selected from surgery, radiation, chemotherapy, immunotherapy, hormone therapy, and virotherapy.

86. A method of treating cancer in a subject in need thereof, the method comprising administering to the subject an effective amount of a combination of (i) a recombinant adenovirus and (ii) an anti-angiogenic agent to treat the cancer in the subject.

87. The method of claim 86, wherein the recombinant adenovirus is a type 5 adenovirus.

88. The method of claim 86 or 87, wherein the recombinant adenovirus does not comprise a deletion of a Pea3 binding site, or a functional fragment thereof.

89. The method of any one of claims 86-88, wherein the recombinant adenovirus comprises an E1a promoter having a deletion of a functional TATA box.

90. The method of claim 89, wherein the deletion comprises a deletion of the entire TATA box.

91. The method of claim 89 or 90, wherein the deletion comprises a deletion of nucleotides corresponding to −27 to −24 of the E1a promoter.

92. The method of any one of claims 89-91, wherein the deletion comprises a deletion of nucleotides corresponding to −31 to −24 of the E1a promoter.

93. The method of any one of claims 89-92, wherein the deletion comprises a deletion of nucleotides corresponding to −44 to +54 of the E1a promoter.

94. The method of any one of claims 89-93, wherein the deletion comprises a deletion of nucleotides corresponding to −146 to +54 of the E1a promoter.

95. The method of any one of claims 89-94, wherein the deletion comprises a deletion of nucleotides corresponding to 472 to 475 of the Ad5 genome (SEQ ID NO: 1).

96. The method of any one of claims 89-95, wherein the deletion comprises a deletion of nucleotides corresponding to 468 to 475 of the Ad5 genome (SEQ ID NO: 1).

97. The method of any one of claims 89-96, wherein the deletion comprises a deletion of nucleotides corresponding to 455 to 552 of the Ad5 genome (SEQ ID NO: 1).

98. The method of any one of claims 89-97, wherein the deletion comprises a deletion of nucleotides corresponding to 353-552 of the Ad5 genome (SEQ ID NO: 1).

99. The method of any one of claims 89-98, wherein the adenovirus comprises a polynucleotide deletion that results in an adenovirus comprising the sequence CTAGGACTG (SEQ ID NO: 23), AGTGCCCG (SEQ ID NO: 30) and/or TATTCCCG (SEQ ID NO: 31).

100. The method of any one of claims 89-99, wherein the E1a promoter comprises the sequence CTAGGACTG (SEQ ID NO: 23).

101. The method of any one of claims 86-100, wherein the recombinant adenovirus comprises an E1a promoter having a deletion of a functional CAAT box.

102. The method of claim 101, wherein the deletion comprises a deletion of the entire CAAT box.

103. The method of claim 101 or 102, wherein the deletion comprises a deletion of nucleotides corresponding to −76 to −68 of the E1a promoter.

104. The method of any one of claims 101-103, wherein the deletion comprises a deletion of nucleotides corresponding to 423 to 431 of the Ad5 genome (SEQ ID NO: 1).

105. The method of any one of claims 101-104, wherein the adenovirus comprises a polynucleotide deletion that results in an adenovirus comprising the sequence TTCCGTGGCG (SEQ ID NO: 32).

106. The method of any one of claims 86-105, wherein the recombinant adenovirus selectively replicates in a hyperproliferative cell.

107. The method of any one of claims 86-106, wherein the recombinant adenovirus selectively expresses endostatin and/or angiostatin in a hyperproliferative cell.

108. The method of claim 106 or 107, wherein the hyperproliferative cell is a cancer cell.

109. The method of any one of claims 86-108, wherein the recombinant adenovirus is an oncolytic adenovirus.

110. The method of any one of claims 86-109, wherein the recombinant adenovirus and anti-angiogenic agent are administered in combination with one or more therapies selected from surgery, radiation, chemotherapy, immunotherapy, hormone therapy, and virotherapy.

111. The method of any one of claims 83-110, wherein the cancer is selected from anal cancer, basal cell carcinoma, bladder cancer, bone cancer, brain cancer, breast cancer, carcinoma, cholangiocarcinoma, cervical cancer, colon cancer, colorectal cancer, endometrial cancer, gastroesophageal cancer, gastrointestinal (GI) cancer, gastrointestinal stromal tumor, hepatocellular carcinoma, gynecologic cancer, head and neck cancer, hematologic cancer, kidney cancer, leukemia, liver cancer, lung cancer, lymphoma, melanoma, merkel cell carcinoma, mesothelioma, neuroendocrine cancer, non-small cell lung cancer, ovarian cancer, pancreatic cancer, pediatric cancer, prostate cancer, renal cell carcinoma, sarcoma, skin cancer, small cell lung cancer, squamous cell carcinoma of the skin, stomach cancer, testicular cancer and thyroid cancer.

112. The method of any one of claims 83-111, wherein the cancer is selected from melanoma, squamous cell carcinoma of the skin, basal cell carcinoma, head and neck cancer, breast cancer, anal cancer, cervical cancer, non-small cell lung cancer, mesothelioma, small cell lung cancer, renal cell carcinoma, prostate cancer, gastroesophageal cancer, colorectal cancer, testicular cancer, bladder cancer, ovarian cancer, liver cancer, hepatocellular carcinoma, cholangiocarcinoma, brain and central nervous system cancer, thyroid cancer, endometrial cancer, neuroendocrine cancer, lymphoma (e.g., Hodgkin and non-Hodgkin), leukemia, merkel cell carcinoma, gastrointestinal stromal tumors, multiple myeloma, uterine cancer, a sarcoma, kidney cancer, ocular cancer, and pancreatic cancer.

113. The method of any one of claims 83-110, wherein the cancer is selected from gastroesophageal cancer (e.g., gastric or gastro-esophageal junction adenocarcinoma), non-small cell lung cancer (e.g., metastatic NSCLC), colorectal cancer (e.g., metastatic colorectal cancer), ovarian cancer (e.g., platinum-resistant ovarian cancer), leukemia, cervical cancer (e.g., late-stage cervical cancer) brain and central nervous system cancer (e.g., glioblastoma), kidney cancer (e.g., renal cell carcinoma), a sarcoma (e.g., rhabdomyosarcoma, osteosarcoma, and Ewing sarcoma), lymphoma (e.g., Hodgkin and non-Hodgkin), ocular cancer (e.g., choroidal melanoma and retinoblastoma), and von Hippel-Lindau disease.

114. The method of any one of claims 83-110, wherein the cancer is selected from brain and central nervous system cancer (e.g., astrocytoma, brain stem glioma, craniopharyngioma, desmoplastic infantile ganglioglioma, ependymoma, high-grade glioma, medulloblastoma, atypical teratoid rhabdoid tumor, neuroblastoma), kidney cancer (e.g., Wilms tumor), ocular cancer (e.g., retinoblastoma), a sarcoma (e.g., rhabdomyosarcoma, osteosarcoma, and Ewing sarcoma), liver cancer (e.g., hepatoblastoma and hepatocellular carcinoma), lymphoma (e.g., Hodgkin and non-Hodgkin), leukemia, and a germ cell tumor.

115. The method of any one of claims 84-114, wherein the anti-angiogenic agent is selected from aflibercept, an anti-VEGF antibody (e.g., bevacizumab and ranibizumab), sunitinib, pazopanib, sorafenib, regorafenib, vandetanib, cabozantinib, axitinib, tivozanib, linifanib, pegaptanib, spironolactone, indomethacin, thalidomide, interleukin-12, an anti-FGF antibody, a tyrosine kinase inhibitor, an interferon, suramin, a suramin analog, somatostatin, and a somatostatin analog.

116. The method of any one of claims 84-114, wherein the anti-angiogenic agent is selected from aflibercept, bevacizumab, ranibizumab, sunitinib, pazopanib, sorafenib, regorafenib, vandetanib, cabozantinib, axitinib, tivozanib and linifanib.

117. The method of claim 115 or 116, wherein the anti-angiogenic agent is bevacizumab.

118. The method of claim 117, wherein bevacizumab is administered at a dose of less than about 5 mg/kg, e.g., from about 1 mg/kg to about 5 mg/kg.

119. The method of claim 118, wherein the bevacizumab is administered at a dose of about 2.5 mg/kg.

120. The method of any one of claims 83-119, wherein the recombinant adenovirus is administered in combination with a second recombinant adenovirus.

121. The method of claim 120, wherein the second recombinant adenovirus comprises a nucleotide sequence encoding a polypeptide, or a fragment thereof, selected from acetylcholine, an androgen-receptor, an anti-PD-1 antibody heavy chain and/or light chain, an anti-PD-L1 antibody heavy chain and/or light chain, BORIS/CTCFL, BRAF, CD19, CD20, CD30, CD80, CD86, CD137, CD137L, CD154, CEA, DKK1/Wnt, EGFRvIII, FGF, gp100, Her-2/neu, ICAM, IL-1, IL-3, IL-4, IL-5, IL-6, IL-8, IL-9, IL-17, IL-23A/p19, p40, IL-24, IL-27, IL-27A/p28, IL-27B/EBI3, IL-35, interferon-gamma, KRAS, MAGE, MAGE-A3, MART1, melan-A, mesothelin, MUC-1, NY-ESO-1, Podocalyxin (Podxl), p53, TGF-β, a TGF-β trap, thymidine kinase, and tyrosinase.

122. The method of claim 120, wherein the second recombinant adenovirus comprises a nucleotide sequence encoding a polypeptide, or a fragment thereof, selected from acetylcholine, an androgen-receptor, an anti-PD-1 antibody heavy chain and/or light chain, an anti-PD-L1 antibody heavy chain and/or light chain, BORIS/CTCFL, BRAF, CD19, CD20, CD30, CD80, CD86, CD137, CD137L, CD154, CEA, DKK1/Wnt, EGFRvIII, FGF, gp100, Her-2/neu, ICAM, IL-1, IL-3, IL-4, IL-5, IL-6, IL-8, IL-9, IL-23A/p19, p40, IL-24, interferon-gamma, KRAS, MAGE, MAGE-A3, MART1, melan-A, mesothelin, MUC-1, NY-ESO-1, Podocalyxin (Podxl), p53, TGF-β, a TGF-β trap, thymidine kinase, and tyrosinase.

123. The method of claim 120, wherein the second recombinant adenovirus comprises a nucleotide sequence encoding a cancer antigen derived from 9D7, androgen receptor, a BAGE family protein, β-catenin, BING-4, BRAF, BRCA1/2, a CAGE family protein, calcium-activated chloride channel 2, CD19, CD20, CD30, CDK4, CEA, CML66, CT9, CT10, cyclin-B1, EGFRvIII, Ep-CAM, EphA3, fibronectin, a GAGE family protein, gp100/pme117, Her-2/neu, HPV E6, HPV E7, Ig, immature laminin receptor, a MAGE family protein (e.g., MAGE-A3), MART-1/melan-A, MART2, MC1R, mesothelin, a mucin family protein (e.g., MUC-1), NY-ESO-1/LAGE-1, P.polypeptide, p53, podocalyxin (Podxl), PRAIVIE, a ras family proteins (e.g., KRAS), prostate specific antigen, a SAGE family protein, SAP-1, SSX-2, survivin, TAG-72, TCR, telomerase, TGF-βRII, TRP-1, TRP-2, tyrosinase, or a XAGE family protein.

124. The method of any one of claims 120-123, wherein the second recombinant adenovirus is an oncolytic adenovirus.

125. A method of lowering blood pressure in a subject in need thereof, the method comprising administering to the subject an effective amount of the recombinant adenovirus of any one of claims 1-78 to lower blood pressure in the subject.

126. A method of increasing nitric oxide (NO) production in a subject in need thereof, the method comprising administering to the subject an effective amount of the recombinant adenovirus of any one of claims 1-78 to increase nitric oxide (NO) production in the subject.

127. A method of treating and/or preventing hypertension in a subject in need thereof, the method comprising administering to the subject an effective amount of the recombinant adenovirus of any one of claims 1-78 to treat and/or prevent hypertension in the subject.

128. The method of any one of claims 82-127, wherein the subject is receiving or has received a VEGF inhibitor.

129. The method of any one of claims 80-128, wherein the effective amount of the recombinant adenovirus is 10²-10¹⁵ plaque forming units (pfus).

130. The method of any one of claims 82-129, wherein the subject is a human or an animal.

131. The method of claim 130, wherein the subject is a pediatric human.


SEQUENCE LISTING (34 sequences)

SEQ ID NO: 1 - DNA, 35938 bp, Adenovirus type 5
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 2 - DNA, 8 bp, Adenovirus type 5
ctgacctc

SEQ ID NO: 3 - DNA, 8 bp, Adenovirus type 5
tcaccagg

SEQ ID NO: 4 - DNA, 8 bp, Adenovirus type 5
cagtatga

SEQ ID NO: 5 - DNA, 10 bp, Adenovirus type 5
taataaaaaa

SEQ ID NO: 6 - protein, 1516 aa, Homo sapiens
MAPYPCGCHILLLLFCCLAAARANLLNLNWLWFNNEDTSHAATTIPEPQGPLPVQPTADTTTHVTPRNGSTEPATAPGSPEPPSELLEDGQDTPTSAESPDAPEENIAGVGAEILNVAKGIRSFVQLWNDTVPTESLARAETLVLETPVGPLALAGPSSTPQENGTTLWPSRGIPSSPGAHTTEAGTLPAPTPSPPSLGRPWAPLTGPSVPPPSSERISEEVGLLQLLGDPPPQQVTQTDDPDVGLAYVFGPDANSGQVARYHFPSLFFRDFSLLFHIRPATEGPGVLFAITDSAQAMVLLGVKLSGVQDGHQDISLLYTEPGAGQTHTAASFRLPAFVGQWTHLALSVAGGFVALYVDCEEFQRMPLARSSRGLELEPGAGLFVAQAGGADPDKFQGVIAELKVRRDPQVSPMHCLDEEGDDSDGASGDSGSGLGDARELLREETGAALKPRLPAPPPVTTPPLAGGSSTEDSRSEEVEEQTTVASLGAQTLPGSDSVSTWDGSVRTPGGRVKEGGLKGQKGEPGVPGPPGRAGPPGSPCLPGPPGLPCPVSPLGPAGPALQTVPGPQGPPGPPGRDGTPGRDGEPGDPGEDGKPGDTGPQGFPGTPGDVGPKGDKGDPGVGERGPPGPQGPPGPPGPSFRHDKLTFIDMEGSGFGGDLEALRGPRGFPGPPGPPGVPGLPGEPGRFGVNSSDVPGPAGLPGVPGREGPPGFPGLPGPPGPPGREGPPGRTGQKGSLGEAGAPGHKGSKGAPGPAGARGESGLAGAPGPAGPPGPPGPPGPPGPGLPAGFDDMEGSGGPFWSTARSADGPQGPPGLPGLKGDPGVPGLPGAKGEVGADGVPGFPGLPGREGIAGPQGPKGDRGSRGEKGDPGKDGVGQPGLPGPPGPPGPVVYVSEQDGSVLSVPGPEGRPGFAGFPGPAGPKGNLGSKGERGSPGPKGEKGEPGSIFSPDGGALGPAQKGAKGEPGFRGPPGPYGRPGYKGEIGFPGRPGRPGMNGLKGEKGEPGDASLGFGMRGMPGPPGPPGPPGPPGTPVYDSNVFAESSRPGPPGLPGNQGPPGPKGAKGEVGPPGPPGQFPFDFLQLEAEMKGEKGDRGDAGQKGERGEPGGGGFFGSSLPGPPGPPGPRGYPGIPGPKGESIRGQPGPPGPQGPPGIGYEGRQGPPGPPGPPGPPSFPGPHRQTISVPGPPGPPGPPGPPGTMGASSGVRLWATRQAMLGQVHEVPEGWLIFVAEQEELYVRVQNGFRKVQLEARTPLPRGTDNEVAALQPPVVQLHDSNPYPRREHPHPTARPWRADDILASPPRLPEPQPYPGAPHHSSYVHLRPARPTSPPAHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 7 - protein, 101 aa, Homo sapiens
QPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKD

SEQ ID NO: 8 - protein, 199 aa, Homo sapiens
SSYVHLRPARPTSPPAHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 9 - DNA, 701 bp, Artificial Sequence
  Synthetic
atctgacctcgtcgacatggctccctacccctgtggctgccacatcctgctgctgctcttctgctgcctggcggctgcccgggccagctcctacgtgcacctgcggccggcgcgacccacaagcccacccgcccacagccaccgcgacttccagccggtgctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggacgagctgctgtttcccagctgggaggctctgttctcaggctctgagggtccgctgaagcccggggcacgcatcttctcctttgacggcaaggacgtcctgaggcaccccacctggccccagaagagcgtgtggcatggctcggaccccaacgggcgcaggctgaccgagagctactgtgagacgtggcggacggaggctccctcggccacgggccaggcctcctcgctgctggggggcaggctcctggggcagagtgccgcgagctgccatcacgcctacatcgtgctctgcattgagaacagcttcatgactgcctccaagtagctcgagtcaccaggcg

SEQ ID NO: 10 - DNA, 738 bp, Artificial Sequence
  Synthetic
atgttcttttctcttacagtatgattaaatgagacatggctccctacccctgtggctgccacatcctgctgctgctcttctgctgcctggcggctgcccgggccagctcctacgtgcacctgcggccggcgcgacccacaagcccacccgcccacagccaccgcgacttccagccggtgctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggacgagctgctgtttcccagctgggaggctctgttctcaggctctgagggtccgctgaagcccggggcacgcatcttctcctttgacggcaaggacgtcctgaggcaccccacctggccccagaagagcgtgtggcatggctcggaccccaacgggcgcaggctgaccgagagctactgtgagacgtggcggacggaggctccctcggccacgggccaggcctcctcgctgctggggggcaggctcctggggcagagtgccgcgagctgccatcacgcctacatcgtgctctgcattgagaacagcttcatgactgcctccaagtagggtctcaaagatcttattccctttaactaataaa

SEQ ID NO: 11 - protein, 810 aa, Homo sapiens
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 12 - protein, 83 aa, Homo sapiens
SECKTGNGKNYRGTMSKTKNGITCQKWSSTSPHRPRFSPATHPSEGLEENYCRNPDNDPQGPWCYTTDPEKRYDYCDILECEE

SEQ ID NO: 13 - protein, 81 aa, Homo sapiens
EECMHCSGENYDGKISKTMSGLECQAWDSQSPHAHGYIPSKFPNKNLKKNYCRNPDRELRPWCFTTDPNKRWELCDIPRCT

SEQ ID NO: 14 - protein, 82 aa, Homo sapiens
YQCLKGTGENYRGNVAVTVSGHTCQHWSAQTPHTHNRTPENFPCKNLDENYCRNPDGKRAPWCHTTNSQVRWEYCKIPSCDS

SEQ ID NO: 15 - protein, 82 aa, Homo sapiens
QDCYHGDGQSYRGTSSTTTTGKKCQSWSSMTPHRHQKTPENYPNAGLTMNYCRNPDADKGPWCFTTDPSVRWEYCNLKKCSG

SEQ ID NO: 16 - protein, 82 aa, Homo sapiens
EDCMFGNGKGYRGKRATTVTGTPCQDWAAQEPHRHSIFTPETNPRAGLEKNYCRNPDGDVGGPWCYTTNPRKLYDYCDVPQC

SEQ ID NO: 17 - protein, 453 aa, Homo sapiens
KVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPR

SEQ ID NO: 18 - DNA, 1451 bp, Artificial Sequence
  Synthetic
atctgacctcgtcgacatggaacataaggaagtggttcttctacttcttttatttctgaaatcaggtcaaggaaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagatagctcgagtcaccaggcg

SEQ ID NO: 19 - DNA, 1488 bp, Artificial Sequence
  Synthetic
atgttcttttctcttacagtatgattaaatgagacatggaacataaggaagtggttcttctacttcttttatttctgaaatcaggtcaaggaaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagatagggtctcaaagatcttattccctttaactaataaa

SEQ ID NO: 20 - DNA, 553 bp, Encephalomyocarditis virus
taacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 21 - DNA, 2673 bp, Artificial Sequence
  Synthetic
atctgacctcgtcgacatggctccctacccctgtggctgccacatcctgctgctgctcttctgctgcctggcggctgcccgggccagctcctacgtgcacctgcggccggcgcgacccacaagcccacccgcccacagccaccgcgacttccagccggtgctccacctggttgcgctcaacagccccctgtcaggcggcatgcggggcatccgcggggccgacttccagtgcttccagcaggcgcgggccgtggggctggcgggcaccttccgcgccttcctgtcctcgcgcctgcaggacctgtacagcatcgtgcgccgtgccgaccgcgcagccgtgcccatcgtcaacctcaaggacgagctgctgtttcccagctgggaggctctgttctcaggctctgagggtccgctgaagcccggggcacgcatcttctcctttgacggcaaggacgtcctgaggcaccccacctggccccagaagagcgtgtggcatggctcggaccccaacgggcgcaggctgaccgagagctactgtgagacgtggcggacggaggctccctcggccacgggccaggcctcctcgctgctggggggcaggctcctggggcagagtgccgcgagctgccatcacgcctacatcgtgctctgcattgagaacagcttcatgactgcctccaagtagtaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggaacataaggaagtggttcttctacttcttttatttctgaaatcaggtcaaggaaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagatagctcgagtcaccaggcg

SEQ ID NO: 22 - DNA, 10 bp, Artificial Sequence
  Sequence resulting from TAV-255 deletion
ggtgttttgg

SEQ ID NO: 23 - DNA, 9 bp, Artificial Sequence
  Sequence resulting from exemplary E1A promoter TATA box deletion
ctaggactg

SEQ ID NO: 24 - DNA, 46 bp, Artificial Sequence
  Exempalry modified E1b-19k region
atcttggttacatctgacctcgtcgagtcaccaggcgcttttccaa

SEQ ID NO: 25 - protein, 1774 aa, Mus musculus
MAPDPSRRLCLLLLLLLSCRLVPASADGNSLSPLNPLVWLWPPKTSDSLEGPVSKPQNSSPVQSTENPTTHVVPQDGLTEQQTTPASSELPPEEEEEEDQKAGQGGSPATPAVPIPLVAPAASPDMKEENVAGVGAKILNVAQGIRSFVQLWDEDSTIGHSAGTEVPDSSIPTVLPSPAELSSAPQGSKTTLWLSSAIPSSPDAQTTEAGTLAVPTQLPPFQSNLQAPLGRPSAPPDFPGRAFLSSSTDQGSSWGNQEPPRQPQHLEGKGFLPMTARSSQQHRHSDVHSDIHGHVPLLPLVTGPLVTASLSVHGLLSVPSSDPSGQLSQVAALPGFPGTWVSHVAPSSGTGLSNDSALAGNGSLTSTSRCLPLPPTLTLCSRLGIGHFWLPNHLHHTDSVEVEATVQAWGRFLHTNCHPFLAWFFCLLLAPSCGPGPPPPLPPCRQFCEALEDECWNYLAGDRLPVVCASLPSQEDGYCVFIGPAAENVAEEVGLLQLLGDPLPEKISQIDDPHVGPAYIFGPDSNSGQVAQYHFPKLFFRDFSLLFHVRPATEAAGVLFAITDAAQVVVSLGVKLSEVRDGQQNISLLYTEPGASQTQTGASFRLPAFVGQWTHFALSVDGGSVALYVDCEEFQRVPFARASQGLELERGAGLFVGQAGTADPDKFQGMISELKVRKTPRVSPVHCLDEEDDDEDRASGDFGSGFEESSKSHKEDTSLLPGLPQPPPVTSPPLAGGSTTEDPRTEETEEDAAVDSIGAETLPGTGSSGAWDEAIQNPGRGLIKGGMKGQKGEPGAQGPPGPAGPQGPAGPVVQSPNSQPVPGAQGPPGPQGPPGKDGTPGRDGEPGDPGEDGRPGDTGPQGFPGTPGDVGPKGEKGDPGIGPRGPPGPPGPPGPSFRQDKLTFIDMEGSGFSGDIESLRGPRGFPGPPGPPGVPGLPGEPGRFGINGSYAPGPAGLPGVPGKEGPPGFPGPPGPPGPPGKEGPPGVAGQKGSVGDVGIPGPKGSKGDLGPIGMPGKSGLAGSPGPVGPPGPPGPPGPPGPGFAAGFDDMEGSGIPLWTTARSSDGLQGPPGSPGLKGDPGVAGLPGAKGEVGADGAQGIPGPPGREGAAGSPGPKGEKGMPGEKGNPGKDGVGRPGLPGPPGPPGPVIYVSSEDKAIVSTPGPEGKPGYAGFPGPAGPKGDLGSKGEQGLPGPKGEKGEPGTIFSPDGRALGHPQKGAKGEPGFRGPPGPYGRPGHKGEIGFPGRPGRPGTNGLKGEKGEPGDASLGFSMRGLPGPPGPPGPPGPPGMPIYDSNAFVESGRPGLPGQQGVQGPSGPKGDKGEVGPPGPPGQFPIDLFHLEAEMKGDKGDRGDAGQKGERGEPGAPGGGFFSSSVPGPPGPPGYPGIPGPKGESIRGPPGPPGPQGPPGIGYEGRQGPPGPPGPPGPPSFPGPHRQTVSVPGPPGPPGPPGPPGAMGASAGQVRIWATYQTMLDKIREVPEGWLIFVAEREELYVRVRNGFRKVLLEARTALPRGTGNEVAALQPPLVQLHEGSPYTRREYSYSTARPWRADDILANPPRLPDRQPYPGVPHHHSSYVHLPPARPTLSLAHTHQDFQPVLHLVALNTPLSGGMRGIRGADFQCFQQARAVGLSGTFRAFLSSRLQDLYSIVRRADRGSVPIVNLKDEVLSPSWDSLFSGSQGQLQPGARIFSFDGRDVLRHPAWPQKSVWHGSDPSGRRLMESYCETWRTETTGATGQASSLLSGRLLEQKAASCHNSYIVLCIENSFMTSFSK

SEQ ID NO: 26 - DNA, 707 bp, Artificial Sequence
  Synthetic
atctgacctcgtcgacatggctcccgaccccagcagacgcctctgcctgctgctgctgttgctgctctcctgccgccttgtgcctgccagcgcttatgtgcacctgccgccagcccgccccaccctctcacttgctcatactcatcaggactttcagccagtgctccacctggtggcactgaacacccccctgtctggaggcatgcgtggtatccgtggagcagatttccagtgcttccagcaagcccgagccgtggggctgtcgggcaccttccgggctttcctgtcctctaggctgcaggatctctatagcatcgtgcgccgtgctgaccgggggtctgtgcccatcgtcaacctgaaggacgaggtgctatctcccagctgggactccctgttttctggctcccagggtcaactgcaacccggggcccgcatcttttcttttgacggcagagatgtcctgagacacccagcctggccgcagaagagcgtatggcacggctcggaccccagtgggcggaggctgatggagagttactgtgagacatggcgaactgaaactactggggctacaggtcaggcctcctccctgctgtcaggcaggctcctggaacagaaagctgcgagctgccacaacagctacatcgtcctgtgcattgagaatagcttcatgacctctttctccaaatagctcgagtcaccaggcg

SEQ ID NO: 27 - protein, 812 aa, Mus musculus
MDHKEVILLFLLLLKPGQGDSLDGYISTQGASLFSLTKKQLAAGGVSDCLAKCEGETDFVCRSFQYHSKEQQCVIMAENSKTSSIIRMRDVILFEKRVYLSECKTGIGNGYRGTMSRTKSGVACQKWGATFPHVPNYSPSTHPNEGLEENYCRNPDNDEQGPWCYTTDPDKRYDYCNIPECEEECMYCSGEKYEGKISKTMSGLDCQAWDSQSPHAHGYIPAKFPSKNLKMNYCRNPDGEPRPWCFTTDPTKRWEYCDIPRCTTPPPPPSPTYQCLKGRGENYRGTVSVTVSGKTCQRWSEQTPHRHNRTPENFPCKNLEENYCRNPDGETAPWCYTTDSQLRWEYCEIPSCESSASPDQSDSSVPPEEQTPVVQECYQSDGQSYRGTSSTTITGKKCQSWAAMFPHRHSKTPENFPDAGLEMNYCRNPDGDKGPWCYTTDPSVRWEYCNLKRCSETGGSVVELPTVSQEPSGPSDSETDCMYGNGKDYRGKTAVTAAGTPCQGWAAQEPHRHSIFTPQTNPRAGLEKNYCRNPDGDVNGPWCYTTNPRKLYDYCDIPLCASASSFECGKPQVEPKKCPGRVVGGCVANPHSWPWQISLRTRFTGQHFCGGTLIAPEWVLTAAHCLEKSSRPEFYKVILGAHEEYIRGLDVQEISVAKLILEPNNRDIALLKLSRPATITDKVIPACLPSPNYMVADRTICYITGWGETQGTFGAGRLKEAQLPVIENKVCNRVEYLNNRVKSTELCAGQLAGGVDSCQGDSGGPLVCFEKDKYILQGVTSWGLGCARPNKPGVYVRVSRFVDWIEREMRNN

SEQ ID NO: 28 - DNA, 1457 bp, Artificial Sequence
  Synthetic
atctgacctcgtcgacatggaccacaaggaagtaatccttctgtttctcttgcttctgaaaccaggacaagggaagagagtgtatctgtcagaatgtaagaccggcatcggcaacggctacagaggaacaatgtccaggacaaagagtggtgttgcctgtcaaaagtggggtgccacgttcccccacgtacccaactactctcccagtacacatcccaatgagggactagaagaaaattactgtaggaacccagacaatgatgaacaagggccttggtgctacactacagatccggacaagagatatgactactgcaacattcctgaatgtgaagaagaatgcatgtactgcagtggcgaaaagtatgaggggaaaatctccaagaccatgtctggacttgactgccaggcctgggattctcagagcccacatgctcatggatacatccctgccaaattcccaagcaagaacctgaagatgaattattgccgcaaccctgacggggagccaaggccctggtgcttcacaacagaccccaccaaacgctgggaatactgtgacatcccccgctgcacaacacccccgcccccacccagcccaacctaccaatgtctgaaaggaagaggtgaaaattaccgagggaccgtgtctgtcaccgtgtctgggaaaacctgtcagcgctggagtgagcaaacccctcataggcacaacaggacaccagaaaatttcccctgcaaaaatctggaggagaattactgccggaacccggatggagaaactgctccctggtgctataccactgacagccagctgaggtgggagtactgtgagattccatcctgcgagtcctcagcatcaccagaccagtcagattcctcagttccaccagaggagcaaacacctgtggtccaggaatgctaccagagcgatgggcagagctatcggggtacatcgtccactaccatcacagggaagaagtgccagtcctgggcagctatgtttccacataggcattcgaagacgccagagaacttcccagatgctggcttggagatgaactattgcaggaacccggatggtgacaagggcccttggtgctacaccactgacccgagcgtcaggtgggaatactgcaacctgaagcggtgctcagagacaggagggagtgttgtggaattgcccacagtttcccaggaaccaagtgggccgagcgactctgagacagactgcatgtatgggaatggcaaagactaccggggcaaaacggccgtcactgcagctggcaccccttgccaaggatgggctgcccaggagccccacaggcacagcatcttcaccccacagacaaacccacgggcaggtctggaaaagaattattgccgaaaccccgatggggatgtgaatggtccttggtgctatacaacaaaccctagatgatagctcgagtcaccaggcg

SEQ ID NO: 29 - DNA, 2685 bp, Artificial Sequence
  Synthetic
atctgacctcgtcgacatggctcccgaccccagcagacgcctctgcctgctgctgctgttgctgctctcctgccgccttgtgcctgccagcgcttatgtgcacctgccgccagcccgccccaccctctcacttgctcatactcatcaggactttcagccagtgctccacctggtggcactgaacacccccctgtctggaggcatgcgtggtatccgtggagcagatttccagtgcttccagcaagcccgagccgtggggctgtcgggcaccttccgggctttcctgtcctctaggctgcaggatctctatagcatcgtgcgccgtgctgaccgggggtctgtgcccatcgtcaacctgaaggacgaggtgctatctcccagctgggactccctgttttctggctcccagggtcaactgcaacccggggcccgcatcttttcttttgacggcagagatgtcctgagacacccagcctggccgcagaagagcgtatggcacggctcggaccccagtgggcggaggctgatggagagttactgtgagacatggcgaactgaaactactggggctacaggtcaggcctcctccctgctgtcaggcaggctcctggaacagaaagctgcgagctgccacaacagctacatcgtcctgtgcattgagaatagcttcatgacctctttctccaaatagtaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggaccacaaggaagtaatccttctgtttctcttgcttctgaaaccaggacaagggaagagagtgtatctgtcagaatgtaagaccggcatcggcaacggctacagaggaacaatgtccaggacaaagagtggtgttgcctgtcaaaagtggggtgccacgttcccccacgtacccaactactctcccagtacacatcccaatgagggactagaagaaaattactgtaggaacccagacaatgatgaacaagggccttggtgctacactacagatccggacaagagatatgactactgcaacattcctgaatgtgaagaagaatgcatgtactgcagtggcgaaaagtatgaggggaaaatctccaagaccatgtctggacttgactgccaggcctgggattctcagagcccacatgctcatggatacatccctgccaaattcccaagcaagaacctgaagatgaattattgccgcaaccctgacggggagccaaggccctggtgcttcacaacagaccccaccaaacgctgggaatactgtgacatcccccgctgcacaacacccccgcccccacccagcccaacctaccaatgtctgaaaggaagaggtgaaaattaccgagggaccgtgtctgtcaccgtgtctgggaaaacctgtcagcgctggagtgagcaaacccctcataggcacaacaggacaccagaaaatttcccctgcaaaaatctggaggagaattactgccggaacccggatggagaaactgctccctggtgctataccactgacagccagctgaggtgggagtactgtgagattccatcctgcgagtcctcagcatcaccagaccagtcagattcctcagttccaccagaggagcaaacacctgtggtccaggaatgctaccagagcgatgggcagagctatcggggtacatcgtccactaccatcacagggaagaagtgccagtcctgggcagctatgtttccacataggcattcgaagacgccagagaacttcccagatgctggcttggagatgaactattgcaggaacccggatggtgacaagggcccttggtgctacaccactgacccgagcgtcaggtgggaatactgcaacctgaagcggtgctcagagacaggagggagtgttgtggaattgcccacagtttcccaggaaccaagtgggccgagcgactctgagacagactgcatgtatgggaatggcaaagactaccggggcaaaacggccgtcactgcagctggcaccccttgccaaggatgggctgcccaggagccccacaggcacagcatcttcaccccacagacaaacccacgggcaggtctggaaaagaattattgccgaaaccccgatggggatgtgaatggtccttggtgctatacaacaaaccctagatgatagctcgagtcaccaggcg

SEQ ID NO: 30 - DNA, 8 bp, Artificial Sequence
  Sequence resulting from exemplary E1A promoter TATA box deletion
agtgcccg

SEQ ID NO: 31 - DNA, 8 bp, Artificial Sequence
  Sequence resulting from exemplary E1A promoter TATA box deletion
tattcccg

SEQ ID NO: 32 - DNA, 10 bp, Artificial Sequence
  Sequence resulting from exemplary E1A promoter CAAT box deletion
ttccgtggcg

SEQ ID NO: 33 - DNA, 8 bp, Adenovirus type 5
tgccttaa

SEQ ID NO: 34 - DNA, 11 bp, Adenovirus type 5
taaaaaaaaat